(19) United States
(12) Patent Application Publication
(10) Pub. No.: US 2025/0136959 A1
(43) Pub. Date: May 1, 2025

(54) TRANSCRIPTION REGULATION SYSTEM BASED ON CRISPRI AND CRISPRA, AND ESTABLISHMENT METHOD THEREFOR AND USE THEREOF

(71) Applicant: EAST CHINA UNIVERSITY OF SCIENCE AND TECHNOLOGY, Shanghai (CN)

(72) Inventors: Menghao CAI, Shanghai (CN); Qi LIU, Shanghai (CN); Yuanxing ZHANG, Shanghai (CN)

(21) Appl. No.: 18/681,789

(22) PCT Filed: Aug. 8, 2022
(86) PCT No.: PCT/CN2022/110767

§ 371 Date: Jun. 21, 2024

(30) Foreign Application Priority Data
Aug. 6, 2021 (CN) 202110901314.9

(51) Int. Cl. C12N 9/22; C12N 15/11; C12N 15/64

(52) U.S. Cl. CPC C12N 9/22; C12N 15/111; C12N 15/64; C12N 2310/20; C12N 2800/102

(57) ABSTRACT

A transcription regulation system based on CRISPRi and CRISPRa, and an establishment method therefor and the use thereof. The system is a new transcription regulation system which has high expression intensity, a low leakage level and is flexible and programmable. Provided is a method for achieving high-intensity and low-leakage expression of a gene by means of the transcription regulation system, wherein a high-intensity transcription level is achieved while suppressing background expression by means of synergistic regulation on downstream signaling effect devices by means of CRISPRi and CRISPRa dev ices. The new transcription regulation system can obtain, by means of loading different input promoters, a new expression system for responding to a specific signal, and has application value in the development and establishment of an efficient heterologous protein expression platform and a microbial cell factory.

This application claims priority to Chinese application No. 202110901314.9 filed on Aug. 6, 2021.

### FIELD OF THE INVENTION

The disclosure belongs to the technical field of biotechnology. More specifically, the disclosure relates to a transcription regulation system based on CRISPRi and CRISPRa, and establishment method therefor and use thereof.

### BACKGROUND

Based on high-quality chassis hosts, high-level and controllable production of functional proteins and chemicals through heterologous expression is currently one of the research hotspots and main directions in the field of synthetic biology and metabolic engineering.

The process of gene transcription mediated by promoters is a key step in determining the intensity of gene expression and regulation pattern. Some highly-efficient natural promoters from different hosts have been identified, developed and widely used in academic research and industrial production. However, with the rapid development of the biological industry, limited by scant number of promoters with high qualities and single signal response pattern, the natural transcription system has been difficult to meet the increasingly diverse research and production needs in this field. Therefore, it is necessary to modify the natural transcription system through promoter engineering and transcription factor engineering technology in order to develop a new regulatory system.

However, the modification of the natural transcription system will inevitably interfere with the cell's own regulatory network and genetic background, making it difficult to make breakthroughs in gene transcription intensities and regulatory patterns.

Therefore, it is necessary to develop an efficient and controllable universal transcription system and protein expression platform that can be artificially customized to meet the increasing research and production needs.

### SUMMARY

The aim of the present disclosure is to provide a novel transcription regulation system based on CRISPRi and CRISPRa, and use thereof.

In the first aspect, the present disclosure provides a transcription regulation system based on CRISPRi and CRISPRa, comprising: a signaling effector device comprising a target promoter and a target gene operably linked thereto; a CRISPR interference (CRISPRi) device targeting and interfering the target promoter and decreasing the expression of the target gene driven by the target promoter: a CRISPR activation (CRISPRa) device targeting and activating the target promoter and increasing the expression of the target gene driven by the target promoter.

In a preferable example, the CRISPR interference device comprises: an expression cassette a, expressing the inactivated Cas protein 1 (CRISPR-dCas) based on the CRISPR system; and, an expression cassette h, expressing a guide RNA, namely giRNA, the giRNA guides the inactivated Cas protein 1 to the target promoter region in the signaling effector device; the CRISPR interference device comprises: an expression cassette c, expressing a fused peptide of the inactivated Cas protein 2 based on the CRISPR system and a transcriptional activator; and, an expression cassette d, expressing, a guide RNA, namely gaRNA or craRNA, and the gaRNA or craRNA guides the inactivated Cas protein 2 to the target promoter region in the signaling effector device: wherein, the inactivated Cas protein 1 and the inactivated Cas protein 2 recognize different PAM sequences in the target promoter sequence and are orthogonal to each other (preferably, the “orthogonal” means that the functions are independent, and there is no crosstalk between the elements); the giRNA and gaRNA or craRNA can form giRNA-gaRNA dimer or giRNA-craRNA dimer, and interact with each other to regulate the strength of interference or activation; preferably, the gaRNA or craRNA is complementary to the partial sequence of the giRNA to form a dimer.

In another preferable example, the giRNA comprises a segment a and a Cas protein binding region a, and the segment a is complementary to the target promoter in the signaling effector device; the gaRNA or the craRNA comprises a segment b and a Cas protein binding region b; the segment h is complementary to segment a, or the segment a or b is complementary to the Cas protein binding region a or b.

In another preferable example, the complementarity comprises substantial complementarity, such as 60%, 70%, 80%, 90%, 95% or 98% complementarity of bases.

In another preferable example, the expression cassette a comprises a promoter driving the expression of the inactivated Cas protein 1; preferably, the promoter comprises: a constitutive promoter or an inducible promoter; more preferably, the promoter comprises (but are not limited to): a GAP promoter, a ENO1 promoter, a GPM1 promoter, a ICL1 promoter, a AOX2 promoter, a TEF1 promoter, a PGK1 promoter, a GTH1 promoter, a DAS1 promoter, a FBA2 promoter, a THI11 promoter, a LRA3 promoter; preferably, the promoter in the expression cassette a is different from the target promoter in the signaling effector device.

In another preferable example, in the expression cassette a, the inactivated Cas protein 1 is a Cas protein or a mutant thereof without nuclease activity; preferably, it is dCas9; preferably, the nucleotide sequence of the dCas9 gene is a sequence as shown in SEQ ID NO: 1 or a degenerate sequence thereof.

In another preferable example, the dCas9 gene also comprises: genes with more than 70% (preferably more than 80%; more preferably more than 90%; more preferably more than 93%; more preferably more than 95%; more preferably more than 97%) identity of SEQ ID NO: 1 and encoding the nucleotide sequence of the same functional protein.

In another preferable example, the expression cassette b comprises a promoter driving, the expression of giRNA; preferably, the promoter comprises: a constitutive promoter or an inducible promoter; preferably, the constitutive promoter comprises (but are not limited to): a GAP promoter, a ENO1 promoter, a GPM1 promoter, a TEF1 promoter, a PGK1 promoter; preferably, the inducible promoter comprises (but are not limited to): a rhamnose-inducible promoter, a methanol-inducible promoter, a thiamine-starvation-inducible promoter: more preferably, the rhamnose-inducible promoter comprises (but are not limited to) a LRA3 promoter, and the methanol-inducible promoter comprises (but are not limited to) a DAS1 promoter, a FBA2 promoter, or the thiamine starvation-inducible promoter comprises (but are not limited to) a THI11 promoter; preferably, the promoter in the expression cassette b is different from the target promoter in the signaling effector device.

In another preferable example, in the expression cassette b, the giRNA guides the inactivated Cas protein 1 in the expression cassette a to the target promoter region in the signaling effector device.

In another preferable example, the expression cassette c comprises a promoter driving the expression of the fused peptide with the inactivated Cas protein 2 and the transcriptional activator; preferably, the promoter comprises: a constitutive promoter or an inducible promoter; more preferably, the promoter comprises (but are not limited to): a GAP promoter, a ENO1 promoter, a GPM1 promoter, a ICL1 promoter, a AOX2 promoter, a TEF1 promoter, a PGK1 promoter, a GTH1 promoter, a DAS1 promoter, a FBA2 promoter, a THI11 promoter, a LRA3 promoter; preferably, the promoter in the expression cassette c is different from the target promoter in the signaling effector device.

In another preferable example, in the expression cassette c, the inactivated Cas protein 2 is a Cas protein or a mutant thereof without nuclease activity; preferably, it comprises VRER or dCpf1; preferably, the nucleotide sequence of the VEGF gene is a sequence as shown in SEQ ID NO: 7 or a degenerate sequence thereof, the nucleotide sequence of the dCpf1 gene is a sequence as shown in SEQ ID NO: 8 or a degenerate sequence thereof.

In another preferable example, the VEGF gene also comprises: genes with more than 70% (preferably more than 80%; more preferably more than 90%; more preferably more than 93%; more preferably more than 95%; more preferably more than 97%) identity of SEQ ID NO: 7 and encoding the nucleotide sequence of the same functional protein.

In another preferable example, the dCpf1 gene also comprises: genes with more than 70% (preferably more than 80%; more preferably more than 90%; more preferably more than 93%; more preferably more than 95%; more preferably more than 97%) identity of SEQ ID NO: 8 and encoding the nucleotide sequence of the same functional protein.

In another preferable example, the expression cassette d comprises a promoter driving the expression of gaRNA or craRNA; preferably, the promoter comprises: a constitutive promoter or an inducible promoter; preferably, the constitutive promoter comprises (but are not limited to): a GAP promoter, a ENO1 promoter, a GPM1 promoter, a TEF1 promoter, a PGK1 promoter; preferably, the inducible promoter comprises (but are not limited to): a rhamnose-inducible promoter, a methanol-inducible promoter, a thiamine starvation-inducible promoter: more preferably, the rhamnose-inducible promoter comprises a LRA3 promoter, and the methanol-inducible promoter comprises a DAS1 promoter, a FBA2 promoter, or the thiamine starvation-inducible promoter comprises a THI11 promoter; preferably, the promoter in the expression cassette d is different from the target promoter in the signaling effector device.

In another preferable example, in the expression cassette d, the gaRNA or craRNA guides the inactivated Cas protein 2 in the expression cassette c to the target promoter region in the signaling effector device.

In another preferable example, the transcriptional activator is a transcription factor with the ability to recruit RNA polymerase independently; preferably, it is VP16, VP64 or VPR; preferably, the nucleotide sequence of the VP16 gene is a sequence as shown in SEQ ID NO: 9 or a degenerate sequence thereof.

In another preferable example, the VP16 gene also comprises: genes with more than 70% (preferably more than 80%; more preferably more than 90%; more preferably more than 93%; more preferably more than 95%; more preferably more than 97%) identity of SEQ ID NO: 9 and encoding the nucleotide sequence of the same functional protein.

In another preferable example, the length of the giRNA is 50-300 bases (such as 60, 80, 100, 120, 140, 160, 180, 200, 250 bases; preferably 80-180 bases); preferably the segment a is located at the 5′-terminal of the giRNA, more preferably the length of segment a is 10-50 bases (such as 12, 15, 18, 20, 22, 25, 28, 30, 5, 40, 45 bases; preferably 15-25 bases); preferably, the segment b is located at the 5-terminal of the gaRNA or the 3′ end of the craRNA, and its length is corresponding to the segment a.

In another preferable example, the Cas protein binding region a or the Cas protein binding region h has at least 1 (such as 1-8, more specifically such as 2, 3, 4, 5, 6 or 7) stem-loop in the secondary structure.

In another preferable example, the target promoter comprises a core promoter, the core promoter is a minimal promoter region with basic transcriptional activity; preferably, the target promoter comprises: a AOX1 promoter or a AOX1 core promoter; more preferably, the sequence of the AOX1 core promoter is shown in SEQ ID NO: 28.

In another preferable example, the target promoter comprises an AOX1 promoter or an AOX1 core promoter; the DNA sequences corresponding to giRNA is shown in any one of SEQ ID NO: 2-6 (respectively giRNA_1, giRNA_2, giRNA_3, giRNA_1c, giRNA_1m).

In another preferable example, the DNA sequence corresponding to the segment a is shown at the 1st to 21 st positions in SEQ ID NO: 2, the 1st to 20th positions in SEQ ID NO: 3 or the 1st to 20th positions in SEQ ID NO: 4.

In another preferable example, the DNA sequence corresponding to the Cas protein binding region a is shown at the 22nd to 101st positions in SEQ ID NO: 2 or the 22nd to 101st positions in SEQ ID NO: 6.

In another preferable example, the giRNA can be used alone or in combination.

In another preferable example, the RNA sequence corresponding to gaRNA is shown in any one of SEQ ID NO: 10-12 (respectively gaRNA 1, gaRNA 2, gaRNA 3, preferably, gaRNA 2); preferably the sequence is shown in SEQ ID NO: 11; the RNA sequence corresponding to craRNA is shown in any one of SEQ ID NO: 13-15 (respectively craRNA_1, craRNA_2, craRNA_3, preferably, craRNA_3); preferably the sequence is shown in SEQ ID NO: 15.

In another preferable example, the DNA sequence corresponding to the segment h is shown at the 1st to the 21st positions of SEQ ID NO: 10, the 1st to the 21st positions of SEQ ID NO: 11 or the 1st to the 91st positions of SEQ ID NO: 12 (corresponding to gaRNA); or shown at the 21st to 40th positions in SEQ ID NO: 13, the 21st to 42nd positions in SEQ ID NO: 14 or the 21st to 40th positions in SEQ ID NO: 15 (corresponding to craRNA).

In another preferable example, the DNA sequence corresponding to the Cas protein binding region b is shown at the 22nd to 101st positions in SEQ ID NO: 10 or the 22nd to 101st positions in SEQ ID NO: 11 (corresponding, to gaRNA); or shown at the 1st to 20th positions in SEQ ID NO: 13 (corresponding to craRNA).

In another preferable example, the signaling effector device comprises sequentially operatively linked from 5 to 3′: a gaRNA-binding sequence or a craRNA-binding sequence (comprising a sequence complementary to the sequence of gaRNA or craRNA), a target promoter (comprising a promoter or a core promoter) and a target gene; preferably, the gaRNA-binding sequence or the craRNA-binding sequence can bind to the corresponding gaRNA or craRNA with a template strand or a non-template strand: wherein, the gaRNA-binding sequence is shown in any one of SEQ ID NO: 16-21; the craRNA binding sequence is shown in any one of SEQ ID NO: 22-27.

In another preferable example, the signaling effector device also comprises a signal amplification element and an intermediate promoter activated thereby; preferably, the signal effect device comprises: (a) a target promoter and a signal amplification element driven thereby; and (b) an intermediate promoter that can be activated by the signal amplification element and the target gene expressed thereby; more preferably, the signal strengthen device comprises an artificial transcription activator STA, a hybrid promoter HIP (intermediate promoter) and an HP-driven target gene.

In another preferable example, the STA gene also comprises: genes with more than 70% (preferably more than 80%; more preferably more than 90%; more preferably more than 93%; more preferably more than 95%; more preferably more than 97%) identity of SEQ ID NO: 29 and encoding the nucleotide sequence of the same functional protein.

In another preferable example, the HP promoter has a sequence as shown in SEQ ID NO: 30 or a functional mutant sequence thereof.

In another aspect, the present disclosure provides a use of any one of the transcription regulation systems described above, wherein, it is used for regulating the expression intensity of the target gene; preferably, it comprises weakening the expression of the target gene or enhancing the expression of the target gene.

In another aspect, the present disclosure provides a method for regulating the expression of a target gene, comprising: establishing any one of the transcriptional regulation systems described above, and interfering or activating the expression according to the expected value of expression intensity of the target gene.

In a preferable example, the CRISPR interference device comprises a giRNA as a guide RNA (for example, giRNA_1, with a nucleotide sequence as shown in SEQ ID NO: 2) and a dCas9 as an inactivated Cas protein 1; the CRISPR activation device comprises a gaRNA as a guide RNA (for example, gaRNA_2, with a nucleotide sequence as shown in SEQ ID NO: 11) and a VRER as an inactivated Cas protein 2; when the giRNA and gaRNA are expressed in different intensities (preferably, driven by promoters with different intensities), the target gene is expressed in different intensities (as exemplified in Example 3).

In a preferable example, the CRISPR interference device comprises a giRNA as a guide RNA (for example, giRNA_1) and a dCas9 as an inactivated Cas protein 1; the CRISPR activation device comprises a craRNA as a guide RNA (for example, craRNA_3, with a nucleotide sequence as shown in SEQ ID NO: 15) and a dCpf1 as an inactivated Cas protein 2; when the giRNA and craRNA are expressed in different intensities (preferably, driven by promoters with different intensities), the target gene is expressed in different intensities (as exemplified in Example 4).

In a preferable example, the CRISPR interference device comprises a giRNA as a guide RNA (for example, giRNA_1), with an inducible promoter used for regulating (opening or closing) the expression of giRNA, and a dCas9 as an inactive Cas protein 1; the CRISPR activation device comprises a craRNA as a guide RNA (for example, craRNA_3, with a nucleotide sequence as shown in SEQ ID NO: 15), and a dCpf1 as an inactive Cas protein 2; when giRNA and craRNA are expressed in different intensities (preferably, driven by promoters with different intensities), the target gene is expressed in different intensities: preferably, the inducible promoters comprises (but are not limited to): a rhamnose-inducible promoter, a methanol-inducible promoters, a thiamine-starvation inducible promoter (as exemplified in Example 5, 6 or 7).

In another aspect, the present disclosure provides a kit for regulating the expression of a target gene, wherein it comprises any one of the transcription regulation systems described above.

Other aspects of the present disclosure will be apparent to those skilled in the art based on the disclosure herein.

### BRIEF DESCRIPTION OF DRAWINGS

FIG. 1. A schematic diagram of RNA interactions.

FIG. 2A-B. Design of giRNA in CRISPRi device (A) and its inhibitory effect on PAOX1 (B).

FIG. 3A-C. Principle of CRISPRa device on cPAOX1 activation (A), design of binding strands (B), and effects of activation (C).

FIG. 4A-B. Regulatory principle (A) and regulatory effects (B) of an artificial transcription regulatory system mediated by VRER+gaRNA_2.

FIG. 5 A-B. Regulatory principle (A) and regulatory effects (B) of an artificial transcription regulatory system mediated by dCpf1+craRNA_3.

FIG. 6 A-B. Validation of a rhamnose-suppressing expression system.

FIG. 7. A dose-response curve of a rhamnose-suppressing expression system to rhamnose concentrations,

FIG. 8 A-B. Validation of a methanol-suppressing expression system.

FIG. 9 A-B. Validation of a thiamine-inducible expression system.

### DETAILED DESCRIPTION

After in-depth researches, the present inventors revealed a method for achieving high-intensity and low-leakage expression of a gene by using CRISPRi and CRISPRa. A novel transcriptional regulation system was constructed and obtained by respectively designing and assembling CRISPR interference devices and CRISPR activation devices. Through synergistic regulation on downstream signaling effect devices by means of CRISPRi and CRISPRa devices, a high-intensity transcription level is achieved while suppressing background expression. The novel transcription regulation system in the present disclosure can obtain, by means of loading different input promoters, a new expression system for responding to a specific signal, and has application value in the development and establishment of an efficient heterologous protein expression platform and a microbial cell factory. As used herein, the “promoter” refers to a nucleic acid sequence, usually at the upstream (5′ terminal) of the coding sequence of a gene of interest, capable of directing the transcription of the nucleic acid sequence into mRNA. Generally, a promoter or promoter region provides a recognition site for RNA polymerase and other factors necessary for the proper initiation of transcription. Herein, the promoter or promoter region comprises a functional variant of the promoter, and the variant may be a naturally occurring allelic variant or a non-naturally occurring variant. The variant comprises a substitution variant, a deletion variant, and a insertion variant.

As used herein, the term “constitutive promoter” refers to a type of promoters that under its regulation, the expression of the target gene is basically constant at the same level, with no obvious difference in gene expression in different tissues, organs and developmental stages.

As used herein, the “inducible promoter” can rapidly induce “on” and “off” or “high” and “low” of gene transcription at a specific cell growth stage or under a specific growth environment as required.

According to the source, inducible promoters can be divided into naturally occurring promoters and artificially constructed promoters.

As used herein, the “intermediate promoter” refers to a promoter that can receive a signal from a specific element (such as a signal amplification element) and be activated to drive the expression of a downstream target gene.

As used herein, the “target gene” refers to a gene whose expression can be directed by the target promoter of the present disclosure. In the present disclosure, there is no particular limitation on suitable target genes, which may be structural genes or non-structural genes. For example, the “target gene” comprises but are not limited to: structural genes, genes encoding proteins with specific functions, enzymes, reporter genes (such as green fluorescent protein, luciferase gene or galactosidase gene LacZ). The protein expressed by the “target gene” can be called “target protein”.

As used herein, the “target promoter” refers to a promoter present in the “signaling effector device” of the present disclosure and regulated by the CRISPR interference device and/or the CRISPR activation device of the present disclosure.

As used herein, the “CRISPR interference device” is a construct comprising a suitable expression cassette, targeting, and interfering the target promoter, weakening the expression of target gene driven by the target promoter.

As used herein, the “CRISPR activation device” is a construct comprising a suitable expression cassette, targeting and activating the target promoter, enhancing the expression of target gene driven by the target promoter.

As used herein, the “signaling effector device” is a construct, comprising a target promoter and a target gene linked thereto; the CRISPR interference device or the CRISPR activation device or a fictional molecule formed in combination with each other can act on the target promoter of the “signaling effector device”, thereby regulating the expression of the target gene.

As used herein, “exogenous” or “heterologous” refers to the relationship between two or more nucleic acid sequences or protein sequences from different sources. For example, if a combination of a promoter and a sequence of the target gene is not natural usually, then the promoter is exogenous to the target gene. A particular sequence is “exogenous” to the cell or organism into which it has been inserted.

As used herein, the “expression cassette” refers to a gene expression system comprising all the necessary elements for expressing a target polypeptide, usually comprising the following elements: a promoter, a gene sequence encoding a polypeptide, a terminator; in addition, optionally comprising a signal peptide encoding sequence and so on. These elements are operatively linked.

As used herein, the “operatively linked” refers to the functional spatial arrangement of two or more nucleic acid regions or nucleic acid sequences. For example: the promoter region is placed at a specific position relative to the nucleic acid sequence of the target gene, so that the transcription of the nucleic acid sequence is guided by the promoter region, thus, the promoter region is “operably linked” to the nucleic acid sequence.

As used herein, the inactivated Cas protein is a mutant of the Cas protein with absent endonuclease activities, but retaining an ability of a guide RNA (gRNA) to lead to a specific position in the genome, and an ability of efficiently binding to a specifically targeted DNA under the guidance of the gRNA.

As used herein, the “comprising”, “having” or “including” includes the terms “containing”, “mainly consisting of” and “essentially consisting of”, and “consisting of”; “mainly composed of”, “basically composed of” and “consisting of” belong to the subordinate concepts of “comprising”, “having” or “including”.

As an emerging gene editing technology, CRISPR/Cas has become a tool for research and application in the fields of biological science and biotechnology due to its high efficiency, flexibility, and easy operation. The CRISPRi system and the CRISPRa system based on the nuclease-free mutant dCas protein can respectively interfere or activate the transcription, with some researches has been reported recently; However, there is no mature and reliable method for efficiently utilizing the two systems in this field.

The present disclosure revealed a method for achieving high-intensity and low-leakage expression of a gene by using CRISPRi and CRISPRa. Through synergistic regulation of the CRISPRi and CRISPRa devices on the expression of downstream core promoter, a highly-efficient and rigorous regulation of the transcription can be achieved. More specifically, in the novel transcription regulation system constructed in the present disclosure, on one hand, the dCas protein in the CRISPRi device will bind to giRNA, locating inside the downstream core promoter to interfere the transcription under the guidance of giRNA; on the other hand, the fused protein of dCas and transcription activator in the CRISPRa device will bind to gaRNA or craRNA, then binding to the corresponding gaRNA or craRNA binding sequence upstream of the core promoter under the guidance of the gaRNA or craRNA, so that the transcription activator and the core promoter are spatially close. Thus, the transcription activator can recruit RNA polymerase to bind to the core promoter to initiate the transcription of the target gene. Wherein, the PAM sequences recognized by the dCas protein in the CRISPRi device and the dCas protein in the CRISPRa device are different and orthogonal to each other; the giRNA in the CRISPRi device and the gaRNA or craRNA in the CRISPRa device can combine with each other to form a dimer, thereby interfering the functions of each other.

In the present disclosure, the dCas protein in the CRISPRi device comprises but are not limited to: dCas9. The nucleotide sequence of the dCas9 gene can be shown in SEQ ID NO: 1. The present disclosure also relates to degenerate sequences of the aforementioned polynucleotides.

The present disclosure also relates to variants of the above-mentioned polynucleotides, wherein the variants encode polypeptides or fragments, analogues and derivatives of the polypeptides with the same amino acid sequences encoded by the above-mentioned polynucleotides.

These nucleotide variants comprise substitution variants, deletion variants, and insertion variants. As known in the art, an allelic variant is an alternative form of a polynucleotide, which may be the substitution, deletion, or insertion of one or more nucleotides, but does not substantially alter the function of the polypeptide it encodes. The present disclosure also relates to polynucleotides homologous to the above-mentioned polynucleotides, preferably with more than 70%, more than 80%, more than 90%, more than 93%, more than 95% or more than 97% of the homology. The polypeptides encoded by these polynucleotides also has the same functions as the polypeptides encoded by the above-mentioned polynucleotides.

The giRNA comprises but are not limited to: giRNA_1, giRNA_2 giRNA_3, giRNA_1c, giRNA_1m.

The DNA sequence of giRNA_1 is shown in SEQ ID NO: 2; the DNA sequence of giRNA_2 is shown in SEQ ID NO: 3; the DNA sequence of giRNA_3 is as shown in SEQ ID NO: 4; the DNA sequence of giRNA_1c is shown in SEQ ID NO: 5; the DNA sequence of giRNA_1m is shown in SEQ ID NO: 6. The present disclosure also relates to degenerate sequences of the aforementioned polynucleotides. The present disclosure also refers to variants of the above polynucleotide, wherein the variants comprise substitution variants, deletion variants, and insertion variants. As known in the art, an allelic variant is an alternative form of a polynucleotide, which may be the substitution, deletion, or insertion of one or more nucleotides, but does not substantially alter the function of the RNA it encodes. The present disclosure also relates to polynucleotides homologous to the above-mentioned polynucleotides, preferably with more than 70%, more than 80%, more than 90%, more than 93%, more than 95% or more than 97% of the homology. The RNAs encoded by these polynucleotides also has the same functions as the RNAs encoded by the above-mentioned polynucleotides.

The CRISPRi device also comprises a promoter element that enables the smooth expression of the dCas protein. Any promoter capable of expressing a large amount of dCas proteins can be used in CRISPRi devices. The promoter can be: a constitutive promoter, an inducible promoter, etc. Preferably, the promoters comprise (but are not limited to): a constitutive promoter P_{GAP}. Similarly, suitable terminators are included in the CRISPRi device. These elements are well known to those skilled in the art for constructing gene expression cassettes.

The CRISPRi device also comprises a promoter element that enables the smooth expression of the giRNA. Preferably, the promoters comprise (but are not limited to): a constitutive promoter P_{GAP}. A rhamnose-inducible promoter, a methanol-inducible promoter, a thiamine-starvation-inducible promoter; preferably, the rhamnose-inducible promoter comprises a LRA3 promoter; preferably, the methanol-inducible promoter comprises a DAS1 promoter, a FBA2 promoter; preferably, the thiamine starvation-inducible promoter comprises a THI11 promoter.

In the present disclosure, the dCas proteins in the CRISPR activation device comprise but are not limited to: VRER, dCpf1; the transcription activator comprises but are not limited to: VP16; the gaRNA comprises but are not limited to: gaRNA_1, gaRNA 2, gaRNA_3; the craRNA comprises but are not limited to: craRNA_1, craRNA_2, craRNA_3. In addition, VRER binds to gaRNA, dCpf1 binds to craRNA; gaRNA_1, gaRNA_2, craRNA_1, craRNA_3 binds to giRNA_1; gaRNA_3 binds to giRNA_1c; craRNA_2 binds to giRNA_1m.

The nucleotide sequence of the VRER gene is shown in SEQ ID NO: 7; the nucleotide sequence of the dCpf1 gene is shown in SEQ ID NO: 8; the nucleotide sequence of the VP16 gene is shown in SEQ ID NO: 9; the DNA sequence of the gaRNA 1 is shown in SEQ ID NO: 10; the DNA sequence of the gaRNA 2 is shown in SEQ ID NO: 11; the DNA sequence of the gaRNA_3 is shown in SEQ ID NO: 12; the DNA sequence of the craRNA_1 is shown in SEQ ID NO: 13; the DNA sequence of the craRNA_2 is shown in SEQ ID NO: 14; the DNA sequence of the craRNA_3 is shown in SEQ ID NO: 15. The present disclosure also relates to degenerate sequences of the aforementioned polynucleotides. The present disclosure also refers to variants of the above polynucleotide, wherein the variants comprise substitution variants, deletion variants, and insertion variants. As known in the art, an allelic variant is an alternative form of a polynucleotide, which may be the substitution, deletion, or insertion of one or more nucleotides, but does not substantially alter the function of the RNA it encodes. The present disclosure also relates to polynucleotides homologous to the above-mentioned polynucleotides, preferably with more than 70%, more than 80%, more than 90%, more than 93%, more than 95% or more than 97% of the homology. The RNAs encoded by these polynucleotides also has the same functions as the polypeptides or RNAs encoded by the above-mentioned polynucleotides.

The CRISPRa device also comprises a promoter element that allows the fused polypeptide of dCas and transcriptional activator and gaRNA or craRNA to be expressed smoothly. Any promoter capable of expressing a large amount of the fused polypeptides and gaRNAs or craRNAs can be used in CRISPRi devices. The promoter can be: a constitutive promoter, an Inducible promoter, etc. Preferably, the promoters comprise (but are not limited to): a constitutive promoter P_{GAP}. Similarly, suitable terminators are included in the CRISPRa device. These elements are well known to those skilled in the art for constructing gene expression cassettes.

In the present disclosure, the gaRNA binding sequences comprise but are not limited to: g1, g1r, g2, g2r, g3, g3r; the craRNA binding sequences comprise but are not limited to: cr1, cr1r, cr2, cr2r, cr3, cr3r. Besides, when gaRNA_1 is used as gaRNA, the corresponding gaRNA binding sequence is g1 or g1r; when gaRNA_2 is used as gaRNA, the corresponding gaRNA binding sequence is g2 or g2r; when gaRNA_3 is used as gaRNA, the corresponding gaRNA binding sequence is g3 or g3r; when craRNA_1 is used as craRNA, the corresponding craRNA binding sequence is cr1 or cr1r; when craRNA_2 is used as craRNA, the corresponding craRNA binding sequence is cr2 or cr2r; when craRNA_3 is used as craRNA, the corresponding craRNA binding sequence is cr3 or cr3r.

The nucleotide sequence of the g1 is shown in SEQ ID NO: 16; the nucleotide sequence of the g1r is shown in SEQ ID NO: 17: the nucleotide sequence of the g2 is shown in SEQ ID NO: 18; the nucleotide sequence of the g2r is shown in SEQ ID NO: 19; the nucleotide sequence of the g3 is shown in SEQ ID NO: 20; the nucleotide sequence of the g3r is shown in SEQ ID NO: 21; the nucleotide sequence of the cr1 is shown in SEQ ID NO: 22; the nucleotide sequence of the cr1r is shown in SEQ ID NO: 23; the nucleotide sequence of the cr2 is shown in SEQ ID NO: 24; the nucleotide sequence of the cr2r is shown in SEQ ID NO: 25; the nucleotide sequence of the cr3 is shown in SEQ ID NO: 26: the nucleotide sequence of the cr3r is shown in SEQ ID NO: 27. The present disclosure also relates to degenerate sequences of the aforementioned polynucleotides. The present disclosure also refers to variants of the above polynucleotide, wherein the variants comprise substitution variants, deletion variants, and insertion variants. As known in the art, an allelic variant is an alternative form of a polynucleotide, which may be the substitution, deletion, or insertion of one or more nucleotides, but does not substantially alter the functions. The present disclosure also refers to a polynucleotide homologous to the above-mentioned polynucleotide, preferably the homology is more than 70%, more than 80%, more than 90%, 93%, more than 95% or more than 97%.

As a preferred embodiment of the present disclosure, the core promoter is the AOX1 core promoter. The sequence of the AOX1 core promoter is shown in SEQ ID NO: 28. The present disclosure also relates to degenerate sequences of the aforementioned polynucleotides. The present disclosure also refers to variants of the above polynucleotide, wherein the variants comprise substitution variants, deletion variants, and insertion variants. As known in the art, an allelic variant is an alternative form of a polynucleotide, which may be the substitution, deletion, or insertion of one or more nucleotides, but does not substantially alter the functions. The present disclosure also refers to a polynucleotide homologous to the above-mentioned polynucleotide, preferably the homology is more than 70%, more than 80%, more than 90%, 93%, more than 95% or more than 97%. In addition, other promoters or core promoters can also be used in the present disclosure.

The PAM sequences recognized by different sources and types of dCas proteins may be quite different, laying a foundation for the orthogonal design and collaborative use of the CRISPRi system and the CRISPRa system. The flexible and programmable performance of gRNA also provides a possibility for using the CRISPR system to develop more complex multifunctional genetic circuits.

The disclosure if further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3^{rd} ed. Science Press, 2002) or followed the manufacturer's recommendation.

### Material

Plasmids were constructed using Seamless Cloning Kit of VazymeBiotech Co. Ltd.

All enzymes used were purchased from TaKaRa Bio Inc. (Dalian, China).

The following commercial plasmids and strains were used for gene cloning and protein expression: plasmids pGAPZ B, pPIC 3.5k, Escherichia coli strain Top10, Pichia pastoris strain GS115, all purchased from invitrogen Corporation; Pichia pastoris strain Δku70 (see patent application No. CN201910403132.1); plasmid pAG32 obtained from the University of California, San Diego; plasmid p414-TEF1p-Cas9-CYC1t acquired from Addgene (43802); plasmid pET28TEV-LbCpf1 obtained from Associate Professor Gao-Yi Tan, East China University of Science and Technology (see Liang M, et al. A CRISPR-Cas12a-derived biosensing platform for the highly sensitive detection of diverse small molecules. Nat Commun. 2019; 10(1):3672).

The plasmid 3.5k-TEF1-gRNA1 was constructed from pPIC3.5k, and the specific method can be found in the publication by Liu Q et al., CRISPR-Cas9-mediated genomic multiloci integration in Pichia pastoris. Microb Cell Fact. 2019; 18(1):144.

The plasmid pDTg1PGAPdCas9 can also be referenced in the publication by Liu Q et al., CRISPR-Cas9-mediated genomic multiloci integration in Pichia pastoris. Microb Cell Fact. 2019; 18(1):144.

The plasmid pPAG was obtained by inserting the GFP gene (full length 714 bp, sequence available under GenBank accession number AY656807.1 from position 80 to 793) at the restriction site of SnaB 1, downstream of the AOX1 promoter site of the plasmid pPIC 3.5k.

The DNA fragments of STA peptide, VP16 peptide, HP promoter, giRNA_1, giRNA_2, giRNA_3, giRNA_4, giRNA_5, giRNA_6, gaRNA 1, gaRNA 2, gaRNA 3, craRNA_1, craRNA_2, and craRNA_3 were all synthesized by GeneWiz Biotechnology Co., Ltd.

Wherein, the DNA sequence of STA peptide is shown in SEQ ID NO:29;
the DNA sequence of VP16 peptide is shown in SEQ ID NO:9;
the sequence of HP promoter is shown in SEQ ID NO:30;
the DNA sequence of giRNA_1 is shown in SEQ ID NO:2;
the DNA sequence of giRNA_2 is shown in SEQ ID NO:3;
the DNA sequence of giRNA_3 is shown in SEQ ID NO:4;
the DNA sequence of giRNA_4 is shown in SEQ ID NO:31;
the DNA sequence of giRNA_5 is shown in SEQ ID NO:32;
the DNA sequence of giRNA_6 is shown in SEQ ID NO:33;
the DNA sequence of gaRNA_1 is shown in SEQ ID NO:10
the DNA sequence of gaRNA_2 is shown in SEQ ID NO: 11;
the DNA sequence of gaRNA_3 is shown in SEQ ID NO:12;
the DNA sequence of craRNA_1 is shown in SEQ ID NO:13;
the DNA sequence of craRNA_2 is shown in SEQ ID NO: 14;
the DNA sequence of craRNA_3 is shown in SEQ ID NO:15.

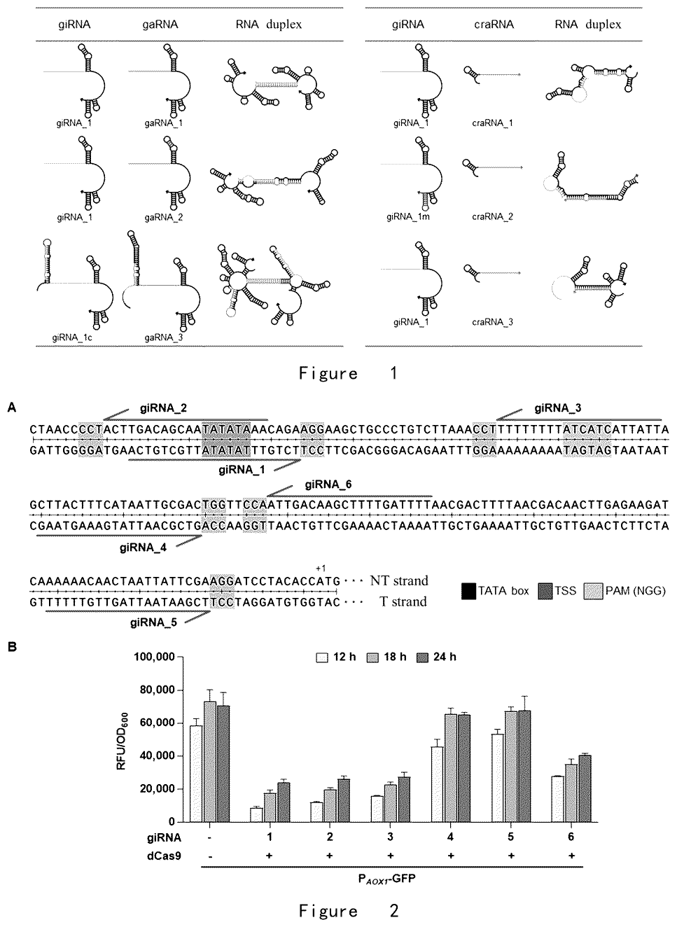

Representative secondary structures formed by giRNA, gaRNA, and craRNA are shown in FIG. 1. The DNA binding region of giRNA was annotated in yellow, while for gaRNA and craRNA, the DNA binding region was annotated in pink. For gaRNA 2, red region on the stem-loop structure represents the mutant region different from the natural sequence. For giRNA_1m, red stein-loop region at the 3′ terminal represents the mutant region different from the natural sequence. The purpose of the mutation is to form a longer binding sequence for dimerization. For giRNA_1c, after adding a stem-loop structure at the 5′ terminal, the dimerization occurs by the binding of giRNA_1c and the 5′ terminal of gaRNA 3. The formation of dimerization will block the binding of guide RNA to the corresponding Cas protein or recognition of specific DNA sites. The secondary structure of the complex in the figure is a predicted structure, and may vary depending on different sequence designs or operational environments, but serves the same function.

The formulations of each medium are as follows:
YPD medium: 2% peptone, 1% yeast extract, 2% glucose.
YPD medium: 2% peptone, 1% yeast extract, 2% glycerol,
YPD medium: 2% peptone, 1% yeast extract, 2% rhamnose.
YND medium: 1% glucose, 0.67% YNB.
YNE medium: 0.5% ethanol, 0.67% YNB.
YNM medium: 0.5% methanol, 0.67% YNB.
Synthetic medium: 2% glycerol, 2% (NH₄)₂SO₄, 1.2% KH₂PO₄, 0.47% MgSO₄·7H₂O, 0.036% CaCl₂, plus trace elements: 0.2 μmol/L CaSO₄·5H₂O, 1.25 μmol/L NaI, 4.5 μmol/L MnSO₄·4H₂O, 2 μmol/L Na₂MoO₄·2H₂O, 0.75 μmol/L H₃BO₃, 17.5 μmol/L ZnSO₄·7H₂O, 44.5 μmol/L FeCl₃·6H₂O, pH 5.5.

When preparing the above media, glucose, glycerol, rhamnose, and trace elements are prepared separately and added when in use. Glucose was sterilized at 115° C. under high pressure for 20 minutes. The trace elements were prepared in solution and then sterilized by filtration. Other components were sterilized at 121° C. under high pressure for 20 minutes. Methanol and ethanol were added when in use. Solid media are supplemented with 2% agar.

Sequence information

SEQ ID NO: 1 (dCas9):
atggacaagaagtactccattgggctcgctatcggcacaaacagcgtcggttgggccgtcattacggacgagtacaaggtgccgagcaaaaaattcaaagttctgggcaataccg
atcgccacagcataaagaagaacctcattggcgccctcctgttcgactccggggagacggccgaagccacgcggctcaaaagaacagcacggcgcagatatacccgcagaaa
gaatcggatctgctacctgcaggagatctttagtaatgagatggctaaggtggatgactctttcttccataggctggaggagtcctttttggtggaggaggataaaaagcacgagcg
ccacccaatctttggcaatatcgtggacgaggtggcgtaccatgaaaagtacccaaccatatatcatctgaggaagaagcttgtagacagtactgataaggctgacttgcggttgat
ctatctcgcgctggcgcatatgatcaaatttcggggacacttcctcatcgagggggacctgaacccagacaacagcgatgtcgacaaactctttatccaactggttcagacttacaa
tcagcttttcgaagagaacccgatcaacgcatcggagttgacgccaaagcaatcctgagcgctaggctgtccaaatcccggcggctcgaaaacctcatcgcacagctccctgg
ggagaagaagaacggcctgtttggtaatcttatcgccctgtcactcgggctgacccccaactttaaatctaacttcgacctggccgaagatgccaagcttcaactgagcaaagaca
cctacgatgatgatctcgacaatctgctggcccagatcggcgaccagtacgcagacctttttttggcggcaaagaacctgtcagacgccattctgctgagtgatattctgcgagtga
acacggagatcaccaaagctccgctgagcgctagtatgatcaagcgctatgatgagcaccaccaagacttgactttgctgaaggcccttgtcagacagcaactgcctgagaagta
caaggaaattttcttcgatcagtctaaaaatggctacgccggatacattgacggcggagcaagccaggaggaattttacaaatttattaagcccatcttggaaaaaatggacggcac
cgaggagctgctggtaaagcttaacagagaagatctgttgcgcaaacagcgcactttcgacaatggaagcatcccccaccagattcacctgggcgaactgcacgctafcctcagg
cggcaagaggatttctacccctttttgaaagataacagggaaaagattgagaaaatcctcacatttcggataccctactatgtaggccccctcgcccggggaaattccagattcgcg
tggatgactcgcaaatcagaagagaccatcactccctggaacttcgaggaagtcgtggataagggggcctctgcccagtccttcatcgaaaggatgactaactttgataaaaatct
gcctaacgaaaaggtgcttcctaaacactctctgctgtacgagtacttcacagtttataacgagctcaccaaggtcaaatacgtcacagaagggatgagaaagccagcattcctgtc
tggagagcagaagaaagctatcgtggacctcctcttcaagacgaaccggaaagttaccgtgaaacagctcaaagaagactatttcaaaaagattgaatgtttcgactctgttgaaat
cagcggagtggaggatcgcttcaacgcatccctgggaacgtatcacgatctcctgaaaatcattaaagacaaggacttcctggacaatgaggagaacgaggacattcttgaggac
attgtcctcacccttacgttgtttgaagatagggagatgattgaagaacgcttgaaaacttacgctcstctcttcgacgacaaagtcatgaaacagctcaagaggcgccgatatacag
gatgggggcggctgtcaagaaaactgatcaatgggatccgagacaagcagagtggaaagacaatcctggattttcttaagtccgatggatttgccaaccggaacttcatgcagttg
atccatgatgactctctcacctttaaggaggacatccagaaagcacaagtttctggccagggggacagtcttcacgagcacatcgctaatcttgcaggtagcccagctatcaaaaa
gggaatactgcagaccgttaaggtcgtggatgaactcgtcaaagtaatgggaaggcataagcccgagaatatcgttatcgagatggcccgagagaaccaaactacccagaagg
gacagaagaacagtagggaaaggatgaagaggattgaagagggtataaaagaactggggtcccaaatccttaaggaacacccagttgaaaacacccagcttcagaatgagaa
gctctacctgtactacctgcagaacggcagggacatgtacgtggatcaggaactggacatcaatcggctctccgactacgacgtggatgccatcgtgccccagtcttttctcaaag
atgattctattgataataaagtgttgacaagatccgataaaaatagagggaagagtgataacgtcccctcagaagaagttgtcaagaaaatgaaaaattattggcggcagctgctga
acgccaaactgatcacacaacggaagttcgataatctgactaaggctgaacgaggtggcctgtctgagttggataaagccggcttcatcaaaaggcagcttgttgagacacgcca
gatcaccaagcacgtggcccaaattctcgattcacgcatgaacaccaagtacgatgaaaatgacaaactgattcgagaggtgaaagttattactctgaagtctaagctggtctcaga
tttcagaaaggactttcagttttataaggtgagagagatcaacaattaccaccatgcgcatgatgcctacctgaatgcagtggtaggcactgcacttatcaaaaaatatcccaagcttg
aatctgaatttgtttacggagactataaagtgtacgatgttaggaaaatgatcgcaaagtctgagcaggaaataggcaaggccaccgctaagtacttcttttacagcaatattatgaat
tttttcaagaccgagattacactggccaatggagagattcggaagcgaccacttatcgaaacaaacggagaaacaggagaaatcgtgtgggacaagggtagggatttcgcgaca
gtccggaaggtcctgtccatgccgcaggtgaacatcgttaaaaagaccgaagtacagaccggaggcttctccaaggaaagtatcctcccgaaaaggaacagcgacaagctgat
cgcacgcaaaaaagattgggaccccaagaaatacggcggattcgattctcctacagtcgcttacagtgtactggttgtggccaaagtggagaaagggaagtctaaaaaactcaaa
agcgtcaaggaactgctgggcatcacaatcatggagcgatcaagcttcgaaaaaaaccccatcgactttctcgaggcgaaaggatataaagaggtcaaaaaagacctcatcatta
agcttcccaagtactctctctttgagcttgaaaacggccggaaacgaatgctcgctagtgcgggcgagctgcagaaaggtaacgagctggcactgccctctaaatacgttaatttct
tgtatctggccagccactatgaaaagctcaaagggtctcccgaagataatgagcagaagcagctgttcgtggaacaacacaaacactaccttgatgagatcatcgagcaaataag
cgaattctccaaaagagtgatcctcgccgacgctaacctcgataaggtgctttctgcttacaataagcacagggataagcccatcagggagcaggcagaaaacattatccacttgtt
tactctgaccaacttgggcgcgcctgcagccttcaagtacttcgacaccaccatagacagaaagcggtacacctctacaaaggaggtcctggacgccacactgattcatcagtca
attacggggctctatgaaacaagaatcgacctctctcagctcggtggagacagcagggctgactaa
SEQ ID NO: 2 (giRNA_1):
tgacagcaatatataaacagagttttagagctagaaatagcaagttaaaataaggctagtccgttatcaacttgaaaaagtggcaccgagtcggtgctttt
SEQ ID NO: 3 (giRNA_2):
tttatatattgctgtcaagtgttttagagctagaaatagcaagttaaaataaggctagtccgttatcaacttgaaaaagtggcaccgagtcggtgctttt
SEQ ID NO: 4 (giRNA_3):
aataatgatgataaaaaaaagttttagagctagaaatagcaagttaaaataaggctagtccgttatcaacttgaaaaagtggcaccgagtcggtgctttt
SEQ ID NO: 5 (giRNA_1c):
gatacttttcagagagcaatatatattgggttatatcttgctctcagaaatgacagcaatatataaacagagttttagagctagaaatagcaagttaaaataaggctagtccgttatcaac
ttgaaaaagtggcaccgagtcggtgctttt
SEQ ID NO: 6 (giRNA_1m):
tgacagcaatatataaacagagttttagagctagaaatagcaagttaaaataaggctagtccgttatcaacttgaaaaagtgtctgctagtagcagatatt
SEQ ID NO: 7 (VRER):
atggacaagaagtactccattgggctcgctatcggcacaaacagcgtcggttgggccgtcattacggacgagtacaaggtgccgagcaaaaaattcaaagttctgggcaataccg
atcgccacagcataaagaagaacctcattggcgccctcctgttcgactccggggagacggccgaagccacgcggctcaaaagaacagcacggcgcagatatacccgcagaaa
gaatcggatctgctacctgcaggagatctttagtaatgagatggctaaggtggatgactctttcttccataggctggaggagtcctttttggtggaggaggataaaaagcacgagcg
ccacccaatctttggcaatatcgtggacgaggtggcgtaccatgaaaagtacccaaccatatatcatctgaggaagaagcttgtagacagtactgataaggctgacttgcggttgat
ctatctcgcgctggcgcatatgatcaaatttcggggacacttcctcatcgagggggacctgaacccagacaacagcgatgtcgacaaactctttatccaactggttcagattacaa
tcagcttttcgaagagaacccgatcaacgcatccggagttgacgccaaagcaatcctgagcgctaggctgtccaaatcccggcggctcgaaaacctcatcgcacagctccctgg
ggagaagaagaacggcctgtttggtaatcttatcgccctgtcactcgggctgacccccaactttaaatctaacttcgacctggccgaagatgccaagcttcaactgagcaaagaca
cctacgatgatgatctcgacaatctgctggcccagatcggcgaccagtacgcagacctttttttggcggcaaagaacctgtcagacgccattctgctgagtgatattctgcgagtga
acacggagatcaccaaagctccgctgagcgctagtatgatcaagcgctatgatgagcaccaccaagacttgactttgctgaaggcccttgtcagacagcaactgcctgagaagta
caaggaaattttcttcgatcagtctaaaaatggctacgccggatacattgacggcggagcaagccaggaggaattttacaaatttattaagcccatcttggaaaaaatggacggcac
cgaggagctgctggtaaagcttaacagagaagatctgttgcgcaaacagcgcactttcgacaatggaagcatcccccaccagattcacctgggcgaactgcacgctatcctcagg
cggcaagaggatttctacccctttttgaaagataacagggaaaagattgagaaaatcctcacatttcggataccctactatgtaggccccctcgcccggggaaattccagattcgcg
tggatgactcgcaaatcagaagagaccatcactccctggaacttcgaggaagtcgtggataagggggcctctgcccagtccttcatcgaaaggatgactaactttgataaaaatct
gcctaacgaaaaggtgcttcctaaacactctctgctgtacgagtacttcacagtttataacgagctcaccaaggtcaaatacgtcacagaagggatgagaaagccagcattcctgtc
tggagagcagaagaaagctatcgtggacctcctcttcaagacgaaccggaaagttaccgtgaaacagctcaaagaagactatttcaaaaagattgaatgtttcgactctgttgaaat
cagcggagtggaggatcgcttcaacgcatccctgggaacgtatcacgatctcctgaaaatcattaaagacaaggacttcctggacaatgaggagaacgaggacattcttgaggac
attgtcctcacccttacgttgtttgaagatagggagatgattgaagaacgcttgaaaacttacgctcatctcttcgacgacaaagtcatgaaacagctcaagaggcgccgatatacag
gatgggggcggctgtcaagaaaactgatcaatgggatccgagacaagcagagtggaaagacaatcctggattttcttaagtccgatggatttgccaaccggaacttcatgcagttg
atccatgatgactctctcacctttaaggaggacatccagaaagcacaagtttctggccagggggacagtcttcacgagcacatcgctaatcttgcaggtagcccagctatcaaaaa
gggaatactgcagaccgttaaggtcgtggatgaactcgtcaaagtaatgggaaggcataagcccgagaatatcgttatcgagatggcccgagagaaccaaactacccagaagg
gacagaagaacagtagggaaaggatgaagaggattgaagagggtataaaagaactggggtcccaaatccttaaggaacacccagttgaaaacacccagcttcagaatgagaa
gctctacctgtactacctgcagaacggcagggacatgtacgtggatcaggaactggacatcaatcggctctccgactacgacgtggatgccatcgtgccccagtcttttctcaaag
atgattctattgataataaagtgttgacaagatccgataaaaatagagggaagagtgataacgtcccctcagaagaagttgtcaagaaaatgaaaaattattggcggcagctgctga
acgccaaactgatcacacaacggaagttcgataatctgactaaggctgaacgaggtggcctgtctgagttggataaagccggcttcatcaaaaggcagcttgttgagacacgcca
gatcaccaagcacgtggcccaaattctcgattcacgcatgaacaccaagtacgatgaaaatgacaaactgattcgagaggtgaaagttattactctgaagtctaagctggtctcaga
tttcagaaaggactttcagttttataaggtgagagagatcaacaattaccaccatgcgcatgatgcctacctgaatgcagtggtaggcactgcacttatcaaaaaatatcccaagcttg
aatctgaatttgtttacggagactataaagtgtacgatgttaggaaaatgatcgcaaagtctgagcaggaaataggcaaggccaccgctaagtacttcttttacagcaatattatgaat
tttttcaagaccgagattacactggccaatggagagattcggaagcgaccacttatcgaaacaaacggagaaacaggagaaatcgtgtgggacaagggtagggatttcgcgaca
gtccggaaggtcctgtccatgccgcaggtgaacatcgttaaaaagaccgaagtacagaccggaggcttctccaaggaaagtatcctcccgaaaaggaacagcgacaagctgat
cgcacgcaaaaaagattgggaccccaagaaatacggcggattcgtttctcctacagtcgcttacagtgtactggttgtggccaaagtggagaaagggaagtctaaaaaactcaaa
agcgtcaaggaactgctgggcatcacaatcatggagcgatcaagcttcgaaaaaaaccccatcgactttctcgaggcgaaaggatataaagaggtcaaaaaagacctcatcatta
agcttcccaagtactctctctttgagcttgaaaacggccggaaacgaatgctcgctagtgcgcgcgagctgcagaaaggtaacgagctggcactgccctctaaatacgttaatttct
tgtatctggccagccactatgaaaagctcaaagggtctcccgaagataatgagcagaagcagctgttcgtggaacaacacaaacactaccttgatgagatcatcgagcaaataag
cgaattctccaaaagagtgatcctcgccgacgctaacctcgataaggtgctttctgcttacaataagcacagggataagcccatcagggagcaggcagaaaacattatccacttgtt
tactctgaccaacttgggcgcgcctgcagccttcaagtacttcgacaccaccatagacagaaaggagtacaggtctacaaaggaggtcctggacgccacactgattcatcagtca
attacggggctctatgaaacaagaatcgacctcctctcagctcggtggagacagcagggctgac
SEQ ID NO: 8 (dcpf1):
atgagcaagctggagaagttcaccaactgctacagcctgagcaagaccctgagattcaaggccatccccgtgggaaaaacccaggagaacatcgacaacaagagactgctggt
ggaggacgaaaagagagccgaggactacaagggcgtgaagaagctgctggacagatactacctgagcttcatcaacgacgtgctgcacagcatcaagctgaagaacctgaac
aactacatcagcctgttcagaaagaagaccagaaccgagaaggagaacaaggagctggagaacctggagatcaacctgagaaaggagatcgccaaggccttcaagggaaac
gagggctacaagagcctgttcaagaaggacatcatcgagaccatcctgcccgagttcctggatgacaaggacgagatcgccctggtgaacagcttcaacggcttcaccaccgct
ttcacccggcttcttcgacaacagagagaacatgttcagcgaggaggccaagtctacaagcatcgccttcagatgcatcaacgagaacctgaccagatacatcagcaacatggaca
tcttcgagaaggtggacgccatcttcgacaagcacgaggtgcaggagatcaaggagaagatcctgaacagcgactacgacgtggaggacttcttcgagggcgagttcttcaactt
cgtgctgacccaggaaggcatcgacgtgtacaacgccatcatcggcggatttgtgacagagagcggcgagaaaatcaagggcctgaacgagtacatcaacctgtacaaccaga
agaccaagcagaagctgcccaagttcaagcccctgtacaagcaggtgctgagcgacagagagagcctgagcttctatggcgagggctacaccagcgatgaagaggtgctgga
ggtgttcagaaacaccctgaacaagaacagcgagatcttcagcagcatcaagaagctggagaagctgttcaagaacttcgacgagtacagcagcgccggcatctttgtgaaaaa
cggccccgctatcagcacaatcagcaaggacatcttcggcgagtggaacgtgatcagagacaagtggaacgccgagtacgacgacatccacctgaagaagaaggccgtggtg
accgagaaatacgaggacgacagaagaaagagcttcaagaagatcggcagcttcagcctggaacagctgcaagagtacgctgacgctgacctgagcgttgtggagaagctga
aggagatcatcatccagaaggtggacgagatctacaaggtgtacggcagcagcgagaaacttttcgacgccgacttcgtgcttgagaagagcctgaagaagaacgatgccgtg
gtggccatcatgaaggacctgctggacagcgtgaagagcttcgagaactacatcaaggccttcttcggcgaaggcaaggagaccaacagagacgagagcttctacggcgactt
cgtgctggcttacgacatcctgctgaaggtggaccacatctacgacgccatcagaaactacgtgacccagaagccctacagcaaggacaagttcaagctgtacttccagaacccc
cagtttatgggcggatgggacaaggataaggagaccgactacagagccaccatcctgagatacggcagcaagtactacctggccatcatggacaagaagtacgccaagtgcct
gcagaagatcgacaaggacgacgtgaacggcaactacgagaagatcaactacaagctgctgcccggccctaataaaatgctgcccaaggtgttcttcagcaagaagtggatgg
cctactacaaccccagcgaggacatccagaagatctacaagaacggcaccttcaagaagggcgacatgttcaacctgaacgactgccacaagctgatcgacttcttcaaggaca
gcatcagcagataccccaagtggagcaacgcctacgacttcaacttcagcgagaccgagaagtacaaggacatcgccggcttctacagagaagtggaggagcagggatacaa
ggtgagcttcgagagcgccagcaagaaggaggtggacaagctggtggaagagggcaagctgtacatgttccagatctacaacaaggacttcagcgacaagtctcacggaacc
cccaatctgcacaccatgtacttcaagctgctgttcgacgagaacaaccacggccagatcagactttctggaggcgctgaactgttcatgagaagagccagcctgaagaaggaag
agctggtggtgcatcctgccaatagccccatcgctaacaagaaccccgacaaccccaagaaaaccaccaccctgagctacgacgtgtacaaggacaagagattcagcgaggac
cagtacgagctgcatatccccatcgccatcaacaagtgccccaagaacatcttcaagatcaacaccgaggtgagagtgctgctgaagcacgacgacaacccctacgtgatcggc
attgccagaggcgagagaaacctgctgtacatcgtggtggtggacggcaagggaaacatcgtggagcagtacagcctgaacgagatcatcaacaacttcaacggcatcagaat
caagaccgactaccacagcctgctggacaagaaggagaaggagagattcgaggccagacagaactggaccagcatcgagaacatcaaggagctgaaggccggctacattag
ccaggtggtgcacaagatctggagctggtggagaagtacgatgccgtgatcgctctggaggatctgaacagcggcttcaagaacagcagagtgaaggtggagaagcaggtgt
accagaagttcgagaagatgctgatcgacaagctgaactacatggtggacaagaagagcaacccctgtgctacaggcggagctctgaagggataccagatcaccaacaagttc
gagagcttcaagagcatgagcacccagaacggcttcatcttctacatccccgcctggctgacatctaagatcgaccctagcaccggctttgtgaacctgctgaagaccaagtacac
cagcalcgccgacagcaagaagttcatcagcagcttcgacagaatcatgtacgtgcccgaggaggacctgtttgaatttgccctggactacaagaacttcagcagaaccgacgcc
gactacatcaagaagtggaagctgtacagctacggcaacagaatcagaatcttcagaaaccccaagaagaacaacgtgttcgactgggaggaggtgtgtctgacaagcgcctac
aaggagctgttcaacaagtacggcatcaactaccagcagggcgacattagagccctgctgtgcgaacagagcgacaaggccttctacagcagcttcatggccctgatgagcctg
atgctgcagatgagaaacagcatcaccggcagaaccgacgtggacttccttatcagccccgtgaaaaacagcgacggcatcttctacgacagcagaaactacgaggcccagga
gaatgctatcctgcccaagaatgccgatgctaacggcgcttacaacatcgccagaaaggtgctttgggccatcggccagtttaagaaggccgaggacgagaagctggacaaggt
gaagatcgccatcagcaacaaggagtggctggagtatgctcagaccagcgtgaaacac
SEQ ID NO: 9 (VP16):
gctccaccaaccgacgtttctttgggtgacgagttgcacttggacggtgaagatgttgccatggctcatgctgacgctttggacgacttcgacttggacatgttgggtgacggtgatt
ctccaggtccaggtttcactccacacgattctgctccatacggtgctttggacatggccgacttcgagtttgagcagatgttcaccgacgctttgggtattgacgagtacggtggttaa
SEQ ID NO: 10 (gaRNA_1):
tctgtttatatattgctgtcagttttagagctagaaatagcaagttaaaataaggctagtccgttatcaacttgaaaaagtggcaccgagtcggtgctttt
SEQ ID NO: 11 (gaRNA_2):
tagctcttaaagtctgtttatgttttagagtcagaaatgacaagttaaaataaggctagtccgttatcaacttgaaaaagtggcaccgagtcggtgctttt
SEQ ID NO: 12 (gaRNA_3):
cgtcacccaatatatattgctctctgaaaatggtggttaatgaaaattaacttactattttctgacagcaaagaaattgtgctatcagatcgttttagagctagaaatagcaagttaaaata
aggctagtccgttatcaacttgaaaaagtggcaccgagtcggtgctttt
SEQ ID NO: 13 (crarna_1): aatttctactaagtgtagatgcacaaactcggacccactt
SEQ ID NO: 14 (crarna_2): aatttctactaagtgtagatactttttcacattgataacgga
SEQ ID NO: 15 (crarna_3): aatttctactaagtgtagatttgataacggactagcctta
SEQ ID NO: 16 (g1): tctgtttatatattgctgtcaagcg
SEQ ID NO: 17 (g1r): cgcttgacagcaatatataaacaga
SEQ ID NO: 18 (g2): tagctcttaaagtctgtttatcgcg
SEQ ID NO: 19 (g2r): cgcgataaacagactttaagagcta
SEQ ID NO: 20 (g3): agaaattgtgctatcagatcagcg
SEQ ID NO: 21 (g3r): cgctgatctgatagcacaatttct
SEQ ID NO: 22 (cr1): tttagcacaaactcggacccactt
SEQ ID NO: 23 (cr1r): aagtgggtccgagtttgtgctaaa
SEQ ID NO: 24 (cr2): tttgactttttcacattgataacgga
SEQ ID NO: 25 (cr2r): tccgttatcaatgtgaaaaagtcaaa
SEQ ID NO: 26 (cr3): tttgttgataacggactagcctta
SEQ ID NO: 27 (cr3r): taaggctagtccgttatcaacaaa
SEQ ID NO: 28 (AOX1
corc)ctaacccctacttgacagcaatatataaacagaaggaagctgccctgtcttaaacctttttttttatcatcattattagcttactttcataattgcgactggttccaattgacaagctt
ttgattttaacgacttttaacgacaacttgagaagatcaaaaaacaactaattattcgaa
SEQ ID NO: 29 (STA):
atgggtgttaagccagttactttgtatgacgttgctgaatacgctggagtttcctaccaaactgtctctagagttgttaatcaagcttctcatgtctccgctaagactagagagaaggttg
aggctgctatggctgaattgaactatattccaaatagagttgctcagcagttggctggaaagcaatctttgttgattggagtcgctacttcttctttggctttgcatgctccatctcagatt
gttgctgctattaagtccagagctcaccagttcggagcttctcttgttctttctatcgttcagagatctggagttcaggcttgcaacgctgctcttcataacttcttggctcagagagttt
ctggattgattattaattacccattggacgatcaagacgctattgccgttgaggccgcttgtaccaacgtcccagctttgttcttggacgtttccgatcaaactccaattaattctattattt
tttctcacgaggatggaactagattgggagttgaacacttggttgctttgggacatcaacagattgctttgttggctggaccattgtcttccgtttctgctagattgagattggccggatg
gcacaagtacttgaccagaaacagattcaaccaattgctgagagagaggggagattggtctgctatgtctggattccagcagactatgcagatgttgaacgaaggaattgtcccaa
ccgctatgttggtccctaatgaccaaatggctttcggagctatgagagctattactgaatctggattgagagtcggagctgacatttctcttcttcgatatgatgacactgaggattctt
cttgctacattccaccattgactactattaagcaagacttcagattgttcggacagacttctgttcatagattcttgcagttctcccaaggacaacctcttaaaggaaaccaaltcttgcc
agtttctttcgttaagagaaagactactttcgctccaaacactcagactccttccccaagagctttgcctgactctttgatccaattgcctagacaactctctagattggagtctcgaca
aggtggcggcggctctgttaacaactccatgaaggatttcttaggcaagaaaacggtggatggagctgatagtctcaatttggccgtgaatctgcaacaacagcagagtt
caaacacaattgccaatcaatcgctttcctcaattggattggaaagttttggttacggctctggtatcaaaaacgagtttaacttccaagacttgataggttcaaactctgg
cagttcagatccgacattttcagtagacgctgacgaggcccaaaaactcgacatttccaacasagascagtcgtaagagacagaaactaggtttgctgccggtcagcaat
gcaacttcccatttgaacggtttcaatggaatgtccaatggaaagtcacactctttctcttcaccgtctgggactaatgacgatgaactaagtggcttgatgttcaactcac
caagcttcaaccccctcacagttaacgattctaccaacaacagcaaccacaatataggtttgtctccgatgtcatgcttattttctacagttcaagaagcatctcaaaaaa
agcatggaaattccagtagacacttttcatacccatctgggccggaggaccctttggttcaatgagttccaaaaacaggccctcacagccaatggagaaaatgctgtcca
acagggagatgatgcttctaagaacascacagccattcctaaggaccagtcttcgaactcatcgattttcagttcacgttctagtgcagcttctagcaactcaggagacg
atattggaaggatgggcccattctccasaggaccagagattgagttcaactacgattcttttttggaatcgttgaaggcagagtcaccctcttcttcaaagtacaatctgcc
ggaaactttgaaagagtacatgacccttagttcgtctcatctgaatagtcaacactccgacactttggcaaatggcactaacggtaactattctagcaccgtttccaacaa
cttgagcttaagtttgaactccttctctttctctgacaagttctcattgagtccaccaacaatcactgacgccgaaaagttttcattgatgagaaacttcattgacaacatctc
gccatggtttgacacttttgacaataccaaacagtttggaacaaaaattccagttctggccaaaaaatgttcttcattgtactatgccattctggctatatcttctcgtcaaa
gagasaggataaagaaagagcacaatgaaaaaacattgcaatgctaccaatactcactacaacagctcatccctactgttcaaagctcaaataatattgagtacattat
cacatgtattctcctgagtgtgttccacatcatgtctagtgaaccttcaacccagagggacatcattgtgtcattggcasaatacattcaagcatgcaacataaacggattt
acatctaatgacanactggaaaagagtattttctggaactatgtcaatttggatttggctacttgtgcaatcggtgaagagtcaatggtcattccttttagctactgggttaa
agagacaactgactacaagaccattcaagatgtgaagccatttttcaccaagaagactagcacgacaactgacgatgacttggacgatatgtatgccatctacatgctg
tacattagtggtagaatcattaacctgttgaactgcagagatgcgaagctcaattttgagcccaagtgggagtttttgtggaatgaactcastgaatgggaattgaacaa
acccttgacctttcasagtattgttcagttcaaggccaatgacgaatcgcagggcggatcaacttttccaactgttcfattctccaactctcgaagctgttacagtaaccag
ctgtatcatatgagctacatcatcttagtgcagaataaaccacgattatacaaaatcccctttactacagtttctgcttcaatgtcatctccatcggacaacaaagctggga
tgtctgcttccagcacacctgcttcagaccaccacgcttctggtgatcatttgtctccaagaagtgtagagccctctctttcgacaacgttgagccctccgcctaatgcaaa
cggtgcaggtaacaagttccgctctacgctctggcatgccaagcagatctgtgggatttctatcaacaacaaccacaacagcaatctagcagccaaagtgaactcattg
caaccattgtggcacgctggaaagctaattagttccaagtctgaacatacacagttgctgsaactgttgaacsaccttgagtgtgcaacaggctggcctatgaactggaa
gggcaaggagttaattgactactggaatgttgaagaataa
SEQ ID NO: 30 (HP):
tctcatgtttgacagcttatcatcgataagctgactcatgttggtattgtgaaatagacgcagatcgggaacgagctcctcgagtgtgtggaattgtgagcggataacaatttcaca
cagtcgagtgtgtggaattgtgagcggataacaatttcacacagtcgagtgtgtggaattgtgagcggataacaatttcacacagtcgagtgtgtggaattgtgagcgga
taacaatttcacacagtcgagtgtgtggaattgtgagcggataacaatttcacacagggcccctaacccctacttgacagcaatatataaacagaaggaagctgccctgtctt
aaacctttttttttatcatcattattagcttactttcataattgcgactggttccaattgacaagcttttgattttaacgacttttaacgacaacttgagaagatcaaaaaacaactaattattcg
aa
SEQ ID NO: 31 (giRNA_4):
cttactttcataattgcgacgttttagagctagaaatagcaagttaaaataaggctagtccgttatcaacttgaaaaagtggcaccgagtcggtgctttt
SEQ ID NO: 32 (giRNA_5):
aaaaacaactaattattcgagttttagagctagaaatagcaagttaaaataaggctagtccgttatcaacttgaaaaagtggcaccgagtcggtgctttt
SEQ ID NO: 33 (giRNA_6):
aaaatcaaaagcttgtcaatgttttagagctagaaatagcaagttaaaataaggctagtccgttatcaacttgaaaaagtggcaccgagtcggtgctttt
SEQ ID NO: 34 (dcas9-TT F): gagacagcagggctgactaagtcgaccatcatcatcatcatc
SEQ ID NO: 35 (dcas9-gap r): gacctttctcttcttttttggaggagtgcaacccatactagtcgaaatagttgttcaattgattgaaatagggac
SEQ ID NO: 36 (dcas9 F1): caaaaaagaagagaaaggtcatggacaagaagtactccattgggctcgctatcggcacaaacagc
SEQ ID NO: 37 (dcas9 R1): cacgatggcatccacgtcgtagtc
SEQ ID NO: 38 (dcas9 F2): acgacgtggatgccatcgtgcc
SEQ ID NO: 39 (dcas9 R2): ttagtcagccctgctgtctc
SEQ ID NO: 40 (pA-AOX1 F): tccagtgtcgaaaacgagctagatctaacatccaaagacgaaag
SEQ ID NO: 41 (pA-AOX1 R): gcggccgcataggccactagataattagttgttttttgatcttctcaagttgtc
SEQ ID NO: 42 (pAA-GAP F): cgcgccttaattacccggggatccctcgagagatcttttttgtagaaatgtcttggtg
SEQ ID NO: 43 (gi1-GAP R):
ctgtttatatattgctgtcagacgagcttactcgtttcgtcctcacggactcatcagtgacagtctagaggtaccatagttgttcaattgattgaaatagggac
SEQ ID NO: 44 (gi1-TT F):
ggcaccgagtcggtgcttttggccggcatggtcccagcctcctcgctggcgcccggctgggcaacatgcttcggcatggcgaatgggacactagtggatgtcagaatgcc
SEQ ID NO: 45 (pAA-TT R): gaagcttcgtacgctgcaggtcgacaagcttgcacaaacgaacgtctcacttaatcttctgtactctgaag
SEQ ID NO: 46 (gi2-GAP R): acttgacagcaatatataaagacgagcttactcgtttcgt
SEQ ID NO: 47 (handle-tt f):
ggcaccgagtcggtgcttttggccggcatggtcccagcctcctcgctggcgccggctgggcaacatgcttcggcatggc
SEQ ID NO: 48 (gi3-GAP R): ttttttttatcatcattattgacgagcttactcgtttcgt
SEQ ID NO: 49 (gi4-GAP R): gtcgcaattatgaaagtaaggacgagcttactcgtttcgt
SEQ ID NO: 50 (gi5-GAP R): tcgaataattagttgtttttgacgagcttactcgtttcgt
SEQ ID NO: 51 (gi6-GAP R): attgacaagcttttgattttgacgagcttactcgtttcgt
SEQ ID NO: 52 (VP-pG F): ttgacgagtacggtgcttaacatcatcatcatcatcattgagtttg
SEQ ID NO: 53 (dcas9V R): gtaggagaaacgaatccgccgtatttc
SEQ ID NO: 54 (dcas9V F): ggcggattcgtttctcctacagtcgct
SEQ ID NO: 55 (dcas9R R): tctgcagctcgcgcgcactagcgagc
SEQ ID NO: 56 (dcas9R F): tagtgcgcgcgagctgcagaaaggta
SEQ ID NO: 57 (dcas9ER R): gtagacctgtactcctttctgtctat
SEQ ID NO: 58 (dcas9ER F): agaaaggagtacaggtctacaaag
SEQ ID NO: 59 (VP-dcas9 R): gaaacgtcggttggtggagcagagccgccgccaccgtcagccctgctgtctcc
SEQ ID NO: 60 (dcpf1-VP F): ctcagaccagcgtgaaacacggtggcggcggctctgctccaccaaccgac
SEQ ID NO: 61 (dcpf1-GAP R): aacttctccagcttgctcatgacctttctcttcttttttggagg
SEQ ID NO: 62 (dcpf1 F1): atgagcaagctggagaagttc
SEQ ID NO: 63 (dcpf1 R1): tcgcctctggcaatgccgat
SEQ ID NO: 64 (dcpf1 F2): atcggcattgccagaggcgag
SEQ ID NO: 65 (dcpf1 R2): gtgtttcacgctggtctg
SEQ ID NO: 66 (ga1-GAP R): tgacagcaatatataaacagagacgagcttactcgtttcgt
SEQ ID NO: 67 (ga2-GAP R): ataaacagactttaagagctagacgagcttactcgtttcgt
SEQ ID NO: 68 (ga3-GAP R): gcaatatatattgggtgacggacgagcttactcgtttcgt
SEQ ID NO: 69 (DR-GAP R): atctacacttagtagaaattgacgagcttactcgtttcgt
SEQ ID NO: 70 (cra1-TT F):
gcacaaactcggacccacttggccggcatggtcccagcctcctcgctggcgccggctgggcaacatgcttcggcatggc
SEQ ID NO: 71 (cra2-TT F):
actttttcacattgataacggaggccggcatggtcccagcctcctcgctggcgccggctgggcaacatgcttcggcatggc
SEQ ID NO: 72 (cra3-TT F):
ttgataacggactagccttaggccggcatggtcccagcctcctcgctggcgccggctgggcaacatgcttcggcatggc
SEQ ID NO: 73 (g1-cA F): ttatatattgctgtcaagcgctaacccctacttgacagca
SEQ ID NO: 74 (pPcAG R): ctgatgttactgaaggatcagatcacgcat
SEQ ID NO: 75 (pPcAG F): tgatccttcagtaacatcagagattttgag
SEQ ID NO: 76 (g1-pP R): cgcttgacagcaatatataaacagactcgaggagctcgttcccgatctgcgtctatttc
SEQ ID NO: 77 (g1r-cA F): cgcttgacagcaatatataaacagactaacccctacttgacagca
SEQ ID NO: 78 (g1r-pP R): tctgtttatatattgctgtcaagcgctcgaggagctcgttcccgatctgcgtctatttc
SEQ ID NO: 79 (g2-cA F): tagctcttaaagtctgtttatcgcgctaacccctacttgacagca
SEQ ID NO: 80 (g2-pP R): cgcgataaacagactttaagagctactcgaggagctcgttcccgatctgcgtctatttc
SEQ ID NO: 81 (g2r-cA F): cgcgataaacagactttaagagctactaacccctacttgacagca
SEQ ID NO: 82 (g2r-pP R): tagctcttaaagtctgtttatcgcgctcgaggagctcgttcccgatctgcgtctatttc
SEQ ID NO: 83 (g3-cA F): agaaattgtgctatcagatcagcgctaacccctacttgacagca
SEQ ID NO: 84 (g3-pP R): cgctgatctgatagcacaatttctctcgaggagctcgttcccgatctgcgtctatttc
SEQ ID NO: 85 (g3r-cA F): cgctgatctgatagcacaatttctctaacccctacttgacagca
SEQ ID NO: 86 (g3r-pP R): agaaattgtgctatcagatccagcgctcgaggagctcgttcccgatctgcgtctatttc
SEQ ID NO: 87 (cr1-cA F): tttagcacaaactcggacccacttctaacccctacttgacagca
SEQ ID NO: 88 (cr1-pP R): aagtgggtccgagtttgtgctaaactcgaggagctcgttcccgatctgcgtctatttc
SEQ ID NO: 89 (cr1r-cA F): aagtgggtccgagtttgtgctaaactaacccctacttgacagca
SEQ ID NO: 90 (cr1r-pP R): tttagcacaaactcggacccacttctcgaggagctcgttcccgatctgcgtctatttc
SEQ ID NO: 91 (cr2-cA F): tttgactttttcacattgataacggactaacccctacttgacagca
SEQ ID NO: 92 (cr2-pP R): tccgttatcaatgtgaaaaagtcaaactcgaggagctcgttcccgatctgcgtctatttc
SEQ ID NO: 93 (cr2r-cA F): tccgttatcaatgtgaaaaagtcaaactaacccctacttgacagca
SEQ ID NO: 94 (cr2r-pP R): tttgactttttcacattgataacggactcgaggagctcgttcccgatctgcgtctatttc
SEQ ID NO: 95 (cr3-cA F): tttgttgataacggactagccttactaacccctacttgacagca
SEQ ID NO: 96 (cr3-pP R): taaggctagtccgttatcaacaaactcgaggagctcgttcccgatctgcgtctatttc
SEQ ID NO: 97 (cr3t-cA F): taaggctagtccgttatcaacaaactaacccctacttgacagca
SEQ ID NO: 98 (cr3r-pP R): tttgttgataacggactagccttactcgaggagctcgttcccgatctgcgtctatttc
SEQ ID NO: 99 (HAPTg1UP F): ctatgaccatgattacgaattcgagct
SEQ ID NO: 100 (HAPTG1DO R): tgcctgcaggtcgactctag
SEQ ID NO: 101 (HP-GFP F): aaaacaactaattattcgaaggatcctacaccatgggttc
SEQ ID NO: 102 (HP-pP R): ataagctgtcaaacatgagaattaattcttgaagacgaaagggc
SEQ ID NO: 103 (STA-TT F): actggaatgttgaagaataaccgcggcggccgcca
SEQ ID NO: 104 (STA-cA r): gtaactggcttaacacccatggtactagtttcgaataattagttgttttttgatc
SEQ ID NO: 105 (TT-HP F): ttaagtgagatcgagtgtgtggaattgtga
SEQ ID NO: 106 (inOri R): gggagaaaggcggacaggta
SEQ ID NO: 107 (inOri F): tacctgtccgcctttctccc
SEQ ID NO: 108 (HP-TT F): acacactcgatctcacttaatcttctgtactctgaag
SEQ ID NO: 109 (pAA-AOX2 F): ttaattaacccggggatccctcgaggcttaaaggactccatttcctaaaat
SEQ ID NO: 110 (HH-AOX2 R): tcatcagtgacagtctagaggtaccttttctcagttgatttgtttg
SEQ ID NO: 111 (pAA-ICL1 F): ttaattaacccggggatccctcgagtcatctaacactttgtatagcacatc
SEQ ID NO: 112 (HH-ICL1 R): tcatcagtgacagtctagaggtacctcttgatatacttgatactgtgttctttga
SEQ ID NO: 113 (pAA-GPM1 F): ttaattaacccggggatccctcgagccttgggttattagtagtgtccgttatttt
SEQ ID NO: 114 (HH-GPM1 R): tcatcagtgacagtctagaggtacctgtttgtttgtgtaattgaaagttgttac
SEQ ID NO: 115 (pAA-ENO1 F): ttaattaacccggggatccctcgagatgaaagagtgagaggaaagtacct
SEQ ID NO: 116 (HH-ENO1 R): tcatcagtgacagtctagaggtaccttttagatgtagattgttataattgtgtgtttcaa
SEQ ID NO: 117 (pAA-LRA3 F): ttaattaacccggggatccctcgagaactgacagaatgactgactcccta
SEQ ID NO: 118 (HH-LRA3 R): tcatcagtgacagtctagaggtaccatttttaggagataaaaattctggggtaaat
SEQ ID NO: 119 (pAA-DAS1 F): ttaattacccggggatccctcgagaataaaaaaacgttatagaaagaaattggactac
SEQ ID NO: 120 (HH-DAS1 R): tcatcagtgacagtctagaggtacctttgttcgattattctccagataaaatcaac
SEQ ID NO: 121 (pAA-THI11 F): ttaattaacccggggatccclcgagatcttttcagcttcatgtcag
SEQ ID NO: 122 (HH-THI11 R): tcatcagtgacagtctagaggtaccgatgatttattgaagtttccaaagttgag

### Example 1. CRISPRI Device Mediated Inhibition on P_{AOX1}

In this example the strain used is Pichia pastoris GS115, and the main devices are as follows.

| | | Plasmids (main elements of the expression |
|---|---|---|
| | | cassette) |
| CRISPR | dCas9 | pGP_{GAP}dCas9 (GAP promoter-dCas) |
| interference | giRNA | pAA-P_{GAP}gi1, pAA-P_{GAP}gi2, pAA-P_{GAP}gi3, |
| devices | | pAA-P_{GAP}gi4, pAA-P_{GAP}gi5, pAA-P_{GAP}gi6 |
| | | (GAP promoter-giRNA) |
| Effectors | GFP | (AOX1 promoter-GFP) |

Main Methods of Construction were as Follows:
1. Construction of pGP_{GAP}dCas9 Plasmid

Using dCas9-TT F (SEQ ID NO: 34) and dCas9-GAP R (SEQ ID N O: 35) as primers, a GAP promoter, a AOXTT terminator and a plasmid backbone region were amplified from the pGAPZ B plasmid by PCR.

Two fragments of dCas9 were amplified from the p411-TEF1p-Cas9-CYC1t plasmid by PCR using dCas9 F1 (SEQ ID NO: 36) and dCas9 R1 (SEQ ID NO: 37), dCas9 F2 (SEQ ID NO: 38) and dCas9 R2 (SEQ ID NO: 39), as primers separately.

By assembling the above fragments using the Seamless Cloning Kit, a recombinant plasmid pGPGAPdCas9 was obtained.

2. Screening of Electroporated Pichia pastoris and GS_AGdCas9 Strains

A recombinant plasmid pPAG was transformed into Pichia pastoris strain GS115 by electroporation, then the transformants were plated onto YND agar plates lacking histidine and cultured at 30° C. in an incubator for 48-72 hours. Single colonies grown on the plates were picked and transferred into liquid culture medium, incubated at 30° C. with shaking, then genomic DNA was extracted, with the copy number of GFP verified using Real-time PCR. The Pichia pastoris strains with single-copy GFP expression were identified by Real-time PCR and designated as GS_AG.

A recombinant plasmid pGPGAPdCas9 was electroporated into Pichia pastoris strain GS-AG, then the transformants were plated onto YPD agar plates supplemented with Zeocin antibiotic and cultured at 30° C. in an incubator for 48-72 hours. Single colonies grown on the plates were picked and transferred into liquid culture medium, incubated at 30° C. with shaking, then genomic DNA was extracted, with the copy number of dCas9 verified using Real-time PCR. The Pichia pastoris strains with single-copy dCas9 expression were identified by Real-time PCR and designated as GS_AGdCas9.

3. Construction of giRNA Expressive Plasmid

An AOX1 promoter fragment was amplified from the plasmid pPAG using PCR by primer pair of pA-AOX1 F (SEQ ID NO: 40) and pA-AOX1 R (SEQ ID NO: 41). By digesting, linearized fragments were obtained from the plasmid pAG32 with SacI/SpeI. Then the linearized fragments were assembled with the AOX1 promoter fragment by seamless cloning, resulting a recombinant plasmid named pAA.

Using a primer pair of pAA-GAP F (SEQ ID NO: 42) and gi1-GAP R (SEQ ID NO: 43), and a primer pair of gi1-TT F (SEQ ID NO: 44) and pAA-TT R (SEQ ID NO: 45), the GAP promoter region and AOX1 terminator region were amplified from the plasmid pPAG via PCR. The plasmid pAA was linearized by digestion with BamH/SalI. These fragments were seamlessly assembled with the giRNA_1 fragment to generate a recombinant plasmid pAA-PGAPgi1 (comprising a GAP promoter, giRNA_1, and AOX1 terminator as an expression cassette).

The plasmid backbone region was amplified from the plasmid pAA-P_{GAP}gi1 by PCR with primer pair of gi2-GAP R (SEQ ID NO: 46) and handle-TT F (SEQ ID NO: 47). Subsequently, the backbone region was assembled with the giRNA_2 fragment using a seamless cloning kit to obtain the recombinant plasmid named pAA-P_{GAP}gi2.

By similar methods, recombinant plasmids of pAA-P_{GAP}gi3, pAA-P_{GAP}gi4, pAA-P_{GAP}gi5 and pAA-P_{GAP}gi6 were obtained.
Primers used in pAA-P_{GAP}gi3 construction: gi3-GAP R (SEQ ID NO: 48) and handle-TT F (SEQ ID NO: 47);
Primers used in pAA-P_{GAP}gi4 construction: gi4-GAP R (SEQ ID NO: 49) and handle-TT F (SEQ ID NO: 47);
Primers used in pAA-P_{GAP}gi5 construction: gi5-GAP R (SEQ ID NO: 50) and handle-TT F (SEQ ID NO: 47);
Primers used in pAA-P_{GAP}gi4 construction: gi4-GAP R (SEQ ID NO: 51) and handle-IT F (SEQ ID NO: 47).
   4. Screening of Pichia pastoris Strains for CRISPRi Device Interference

Recombinant plasmids pAA-P_{GAP}gi1, pAA-P_{GAP}gi2, pAA-P_{GAP}gi3, pAA-P_{GAP}gi4, pAA-P_{GAP}gi5 and pAA-P_{GAP}gi6 were separately electroporated into Pichia pastoris strain GS_AGdCas9, then the transformants were plated onto YPD agar plates supplemented with Hygromycin antibiotic and cultured at 30° C. in an incubator for 48-72 hours.

Single colonies grown on the plates were picked and transferred into liquid culture medium, incubated at 30° C. with shaking, then the genomic DNA was extracted, with the copy number of giRNA verified using Real-time PCR.

The Pichia pastoris strains with single-copy giRNA expression were identified by Real-time PCR and separately designated as:
GS_AGdCas9-GAPgi1, GS_AGdCas9-GAPgi2, GS_AGdCas9-GAPgi3,
GS_AGdCas9-GAPgi4, GS_AGdCas9-GAPgi5, GS_AGdCas9-GAPgi6.

### 5. Detection of GFP Fluorescence Intensity by Microplate Reader

The strains GS_AG, GS_AGdCas9-GAPgi1, GS_AGdCas9-GAPgi2, GS_AGdCas9-GAPgi3, GS_AGdCas9-GAPgi4, GS_AGdCas9-GAPgi5 and GS_AGdCas9-GAPgi6 were separately pre-cultured overnight in YPD liquid medium, then the strains were collected by centrifugation, washed twice with distilled water and transferred to YNM liquid medium for cultivation. Samples were taken and GFP fluorescence intensity in the samples was detected using a microplate reader.

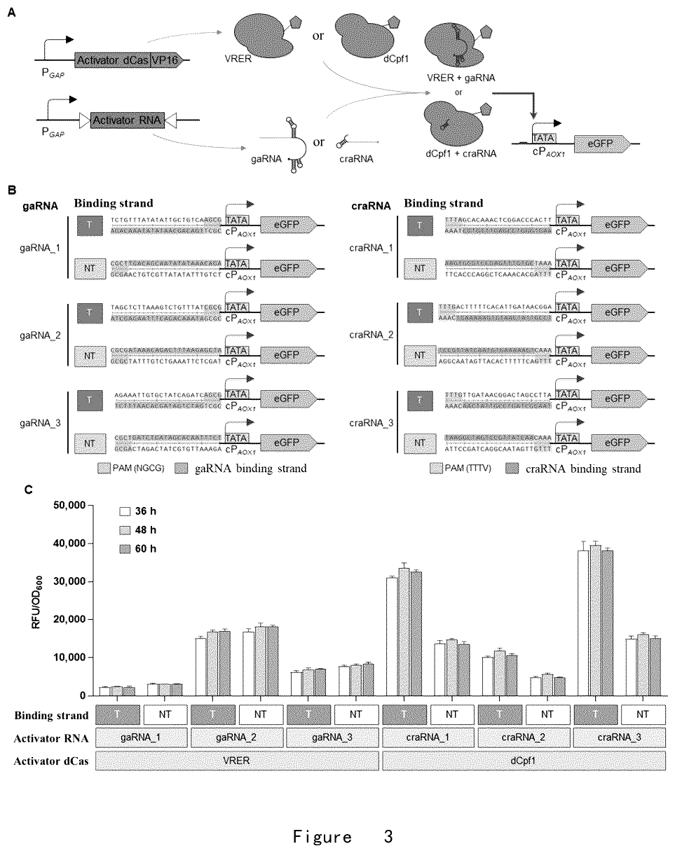

As shown in FIG. 2A and FIG. 2B, compared to the wild-type AOX1 promoter, the fluorescence intensity of each strain was reduced in different degrees after introducing the CRISPR inhibitive device composed of dCas9 and giRNA. Wherein, the CRISPR inhibitive device mediated by giRNA_1 showed the best inhibitive effect on the AOX1 promoter with a reduced expression intensity of 65.9%.

### Example 2. CRISPRa Device Mediated Activation on cP_{AOX1} (AOX1 Core Promoter)

In this example, the strain used is Pichia pastoris GS115, and the main devices are as follows:

| | | Plasmids (main elements of the expression cassette) | Others |
|---|---|---|---|
| CRISPR | VRER | pGP_{GAP}VRERVP16 (GAP promoter-VRER-VP16) | VP16: transcription |
| activation | dCpf1 | pGP_{GAP}dCpf1VP16 (GAP promoter-dCpf1-VP16) | activator |
| devices | gaRNA | pAA-P_{GAP}ga1, pAA-P_{GAP}ga2, pAA-P_{GAP}ga3, pAA- | |
| | craRNA | P_{GAP}cra1, pAA-P_{GAP}cra2, pAA-P_{GAP}cra3 | |
| | | (GAP promoter-gaRNA or GAP promoter-craRNA) | |
| Effectors | GFP | pPg1cAG, pPg1rcAG, pPg2cAG, pPg2rcAG, | gaRNA/craRNA |
| | | pPg3cAG, pPg3rcAG, pPcr1cAG, pPcr1rcAG, | binding sequence: |
| | | pPcr2cAG, pPcr2rcAG, pPcr3cAG, pPcr3rcAG | used to guide the |
| | | (gaRNA/craRNA binding sequence-AOX1 core | targeting of |
| | | promoter-GFP) | gaRNA/craRNA |

Main Methods of Construction were as Follows:
1. Construction of pGP_{GAP}VRERVP16 Plasmid and pGP_{GAP}dCpf1VP16 Plasmid

Several regions of VRER (SEQ ID NO: 7) and plasmid backbone region were amplified from the plasmid pGP_{GAP}dCas9 using PCR with primer pairs of VP-pG F (SEQ ID NO: 52) and dCas9V R (SEQ ID NO: 53), dCas9V F (SEQ ID NO: 54) and dCas9R R (SEQ ID NO: 55), dCas9R F (SEQ ID NO: 56) and dCas9ER R (SEQ ID NO: 57), dCas9ER F (SEQ ID NO: 58) and VP-dCas9 R (SEQ ID NO: 59). By assembling the above fragments with VP16 fragment using the Seamless Cloning Kit, a recombinant plasmid pGP_{GAP}VRERVP16 was obtained.

The backbone region except VEGF was amplified from the plasmid pGP_{GAP}VRERVP16 using PCR with a primer pair of dCpf1-VP F (SEQ ID NO: 60) and dCpf1-GAP R (SEQ ID NO: 61). Two regions of dCpf1 were amplified from the plasmid pET28TEV-LbCpf1 using PCR with primer pairs of dCpf1 F1 (SEQ ID NO: 62) and dCpf1 RI (SEQ ID NO: 63), dCpf1 F2 (SEQ ID NO: 64) and dCpf1 R2 (SEQ ID NO: 65), separately. By assembling the above fragments using the Seamless Cloning Kit, a recombinant plasmid pGP_{GAP}dCpf1 VP16 was obtained.

### 2. Screening of Pichia Pastoris GS_VV Strains and GS_dCV Strains

Recombinant plasmids pGP_{GAP}VRERVP16 and pGP_{GAP}dCpf1VP16 were electroporated separately into Pichia pastoris strain GS 115, then the transformants were plated onto YPD agar plates supplemented with Zeocin antibiotic and cultured at 30° C. in an incubator for 48-72 hours, Single colonies grown on the plates were picked and transferred into liquid culture medium, cultivated in an incubator shaker at 30° C., then the genomic DNA was extracted, with the copy number of VP16 verified using Real-time PCR. The Pichia pastoris strains with single-copy VP16 expression were identified by Real-time PCR and separately designated as GS_VV and GS_dCV.

3. Construction of P_{GAP} Expressive gaRNA Plasmid or craRNA Plasmid

The plasmid backbone region was amplified from the plasmid pAA-P_{GAP}gi1 by PCR with primer pair of gal-GAP R (SEQ ID NO: 66) and handle-TT F (SEQ ID NO: 47). Subsequently, the backbone region was assembled with the gaRNA_1 fragment using a seamless cloning kit to obtain the recombinant plasmid named pAA-P_{GAP}ga1.

By similar methods, recombinant plasmids of pAA-P_{GAP}ga2 (with gaRNA_2 fragment assembled), pAA-P_{GAP}ga3 (with gaRNA_3 fragment assembled), pAA-P_{GAP}cra1 (with craRNA_1 fragment assembled), pAA-P_{GAP}cra2 (with craRNA_2 fragment assembled) and pAA-P_{GAP}cra3 (with craRNA_3 fragment assembled) were obtained.
Primers used in pAA-P_{GAP}ga2 construction: ga2-GAP P (SEQ ID NO: 67) and handle-TT F (SEQ ID NO: 47);
Primers used in pAA-P_{GAP}ga3 construction: ga3-GAP R (SEQ ID NO: 68) and handle-TT F (SEQ ID NO: 47);
Primers used in pAA-P_{GAP}cra1 construction: DR-GAP R (SEQ ID NO: 69) and cra1-TT F (SEQ ID NO: 70);
Primers used in pAA-P_{GAP}cra2 construction: DR-GAP R (SEQ ID NO: 69) and cra2-TT F (SEQ ID NO: 71);
Primers used in pAA-P_{GAP}cra3 construction: DR-GAP R (SEQ ID NO: 69) and cra3-TT F (SEQ ID NO: 72).
   4. Screening of P_{GAP} Expressive gaRNA or craRNA Strains

Recombinant plasmids pAA-P_{GAP}ga1, pAA-P_{GAP}ga2 and pAA-P_{GAP}ga3 were electroporated separately into Pichia pastoris strain GS_VV, then the transformants were plated onto YPD agar plates supplemented with Hygromycin antibiotic and cultured at 30° C. in an incubator for 48-72 hours, Single colonies grown on the plates were picked and transferred into liquid culture medium, incubated at 30° C. with shaking, then the genomic DNA was extracted, with the copy number of gaRNA verified using Real-time PCR. The Pichia pastoris strains with single-copy gaRNA expression were identified by Real-time PCR and separately designated as GS_VV-ga1, GS_VV-ga2 and GS_VV-ga3.

Recombinant plasmids pAA-P_{GAP}cra1, pAA-P_{GAP}cra2 and pAA-P_{GAP}cra3 were electroporated separately into Pichia pastoris strain GS_dCV, then the transformants were plated onto YPD agar plates supplemented with Hygromycin antibiotic and cultured at 30° C. in an incubator for 48-72 hours. Single colonies grown on the plates were picked and transferred into liquid culture medium, incubated at 30° C. with shaking, then the genomic DNA was extracted, with the copy number of craRNA verified using Real-time PR. The Pichia pastoris strains with single-copy gaRNA expression were identified by Real-time PCR and separately designated as GS_VV-cra1, GS_VV-cra2 and GS_VV-cra3, 5. Construction of GFP expressive plasmid A region containing AOX1 core promoter and GFP and the plasmid backbone region (except core promoter and GFP, gaRNA binding sequence and craRNA binding sequence were included) were amplified from the plasmid pPAG by PCR with primer pairs of g1-cA F (SEQ ID NO: 73) and pPcAG R (SEQ ID NO: 74), pPcAG F (SEQ ID NO: 75) and g1-pP R (SEQ ID NO: 76). Subsequently, two regions were assembled using a seamless cloning kit to obtain the recombinant plasmid named pPg1cAG.
By similar methods, pPg1rcAG, pPg2cAG pPg2rcAG, pPg3cAG, pPg3rcAG, pPcr1cAG, pPcr1rcAG pPcr2cAG, pPcr2rcAG, pPcr3cAG, pPcr3rcAG were obtained.
Primers used in pPg1rcAG construction: g1r-cA F (SEQ ID NO: 77) and g1r-pP R (SEQ ID NO: 78);
Primers used in pPg2cAG construction: g2-cA F (SEQ ID NO: 79) and g2-pP R (SEQ ID NO: 80);
Primers used in pPg2rcAG construction: g2r-cA F (SEQ ID NO: 81) and g2r-pP R(SEQ ID NO: 82);
Primers used in pPg3cAG construction: g3-cA F (SEQ ID NO: 83) and g3-pP R (SEQ ID NO: 84);
Primers used in pPg3rcAG construction: g3r-cA F (SEQ ID NO: 85) and g3r-pP R. (SEQ ID NO: 86);
Primers used in pPcr1cAG construction: cr1-cA F (SEQ ID NO: 87) and cr1-pP R (SEQ ID NO: 88);
Primers used in pPcr1rcAG construction: cr1r-cA F (SEQ ID NO: 89) and cr1r-pP R(SEQ ID NO: 90);
Primers used in pPcr2cAG construction: cr2-cA F (SEQ ID NO: 91) and cr2-pP R (SEQ ID NO: 92);
Primers used in pPcr2rcAG construction: cr2r-cA F (SEQ ID NO: 93) and cr2r-pP R(SEQ ID NO: 94);
Primers used in pPcr3cAG construction: cr3-cA F (SEQ ID NO: 95) and cr3-pP R (SEQ ID NO: 96);
Primers used in pPcr3rcAG construction: cr3r-cA F (SEQ ID NO: 97) and cr3r-pP R (SEQ ID NO: 98).
   6. Screening of Pichia pastoris Strains for CRISPR Activation

Recombinant plasmids pPg1cAG and pPg1rcAG were transformed into Pichia pastoris strain GS_VV-gal by electroporation, then the transformants were plated onto YND agar plates lacking histidine and cultured at 30° C. in an incubator for 48-72 hours. Single colonies grown on the plates were picked and transferred into liquid culture medium, incubated at 30° C. with shaking, then genomic DNA was extracted, with the copy number of GFP verified using Real-time PCR. The Pichia pastoris strains with single-copy GFP expression were identified by Real-time PCR and separately designated as GS_VV-ga1-g1cAG and GS_VV-ga1-g1rcAG.

Recombinant plasmids pPg2cAG and pPg2rcAG were transformed into Pichia pastoris strain GS_VV-ga2 by electroporation, then the transformants were plated onto YND agar plates lacking histidine and cultured at 30° C. in an incubator for 48-72 hours. Single colonies grown on the plates were picked and transferred into liquid culture medium, incubated at 30° C. with shaking, then genomic DNA was extracted, with the copy number of GFP verified using Real-time PCR. The Pichia pastoris strains with single-copy GFP expression were identified by Real-time PCR and separately designated as GS_VV-ga2-g2cAG and GS_VV-ga2-g2rcAG.

Recombinant plasmids pPg3cAG and pPg3rcAG were transformed into Pichia pastoris strain GS_VV-ga3 by electroporation, then the transformants were plated onto YND agar plates lacking histidine and cultured at 30° C. in an incubator for 48-72 hours. Single colonies grown on the plates were picked and transferred into liquid culture medium, incubated at 30′C with shaking, then genomic DNA was extracted, with the copy number of GFP verified using Real-time PCR. The Pichia pastoris strains with single-copy GFP expression were identified by Real-time PCR and separately designated as GS_VV-ga3-g3cAG and GS_VV-ga3-g3rcAG.

Recombinant plasmids pPcr1cAG and pPcr1rcAG were transformed into Pichia pastoris strain GS_dCV-cra1 by electroporation, then the transformants were plated onto YND agar plates lacking histidine and cultured at 30° C. in an incubator for 48-72 hours. Single colonies grown on the plates were picked and transferred into liquid culture medium, incubated at 30° C. with shaking, then genomic DNA was extracted, with the copy number of GFP verified using Real-time PCR. The Pichia pastoris strains with single-copy GFP expression were identified by Real-time PCR and separately designated as GS_dCV-cra1-cr1cAG and GS_dCV-cra1-cr1rcAG.

Recombinant plasmids pPcr2cAG and pPcr2rcAG were transformed into Pichia pastoris strain GS_dCV-cra2 by electroporation, then the transformed cells were plated onto YND agar plates lacking histidine and cultured at 30° C. in an incubator for 48-72 hours. Single colonies grown on the plates were picked and transferred into liquid culture medium, incubated at 30° C. with shaking, then genomic DNA was extracted, with the copy number of GFP verified using Real-time PCR. The Pichia pastoris strains with single-copy GFP expression were identified by Real-time PCR and separately designated as GS_dCV-cra2-cr2cA G and GS_dCV-cra2-cr2rcAG.

Recombinant plasmids pPcr3cAG and pPcr3rcAG were transformed into Pichia pastoris strain GS_dCV-cra3 by electroporation, then the transformed cells were plated onto YND agar plates lacking histidine and cultured at 30° C. in an incubator for 48-72 hours. Single colonies grown on the plates were picked and transferred into liquid culture medium, incubated at 390° C. with shaking, then genomic DNA was extracted, with the copy number of GFP verified using Real-time PCR. The Pichia pastoris strains with single-copy GFP expression were identified by Real-time PCR and separately designated as GS_dCV-cra3-cr3cAG and GS_dCV-cra3-cr3rcAG.

### 7. Detection of GFP Fluorescence Intensity by Microplate Reader

The strains GS_VV-ga1-g1cAG, GS_VV-ga1-g1rcAG, GS_VV-ga2-g2cAG, GS_VV-ga2-g2rcAG. GS_VV-ga3-g3cAG, GS_VV-ga3-g3rcAG, GS_dCV-cra1-cr1cAG, GS_dCV-cra1-cr1rcAG, GS_dCV-cra2-cr2cAG, GS_dCV-cra2-cr2rcAG, GS_dCV-cra3-cr3cAG and GS_dCV-cra3-cr3rcAG were separately pre-cultured overnight in YPD liquid medium, centrifuged to collect cells, washed twice with distilled water and then transferred and cultured in a YNM liquid medium. Then samples were taken and the GFP fluorescence intensity in the samples was measured using a microplate reader.

As shown in FIGS. 3A-C, all strains expressed fluorescent proteins. Among them, gaRNA_2 mediated CRISPR activation device functioned the best when using VRER-VP16 as an activator, followed by gaRNA_3, with a certain degree of activating effect. The two can significantly increase the activity of cP_{AOX1}, and a slightly higher eGFP fluorescence intensity was observed when gaRNA bound to the non-template strand (NT).

When using dCpf1-VP16 as the activator, the CRISPR activation devices mediated by craRNA_1 and craRNA_3 both showed better activating effects, and craRNA demonstrated better activation effects when bound to the template strand.

### Example 3. Interference Device (dCas9-+giRNA_1) and Activation Device (VRER+gaRNA_2) Mediated Artificial Transcription Regulatory Systems

In this example, the strain used is Pichia pastoris, Δku70, and the main devices are as follows:

| | | Plasmids (main elements of the expression cassette) | Others |
|---|---|---|---|
| CRISPR | dCas9 | 3.5k-TEF1-gRNA1; dCas9-HA | |
| interference | giRNA_1 | AOX2, ICL1, GPM1, ENO1, GAP separately | |
| devices | | drives giRNA_1 | |
| CRISPR | VRER | pGP_{GAP}VRERVP16 (GAP promoter-VRER-VP16) | VP16: |
| activation | | | Individually |
| devices | | | recruits RNA |
| | | | polymerase |
| | gaRNA_2 | AOX2, ICL1, GPM1, ENO1, GAP separately drives gaRNA_2 | |
| Effectors | GFP | pPg2rcATSAD (HP promoter-GFP and gaRNA_2 binding | STA: signal |
| | | sequence-AOX1 core promoter-STA) | amplification |

Main Methods of Construction were as Follows:
1. Screening of Pichia pastoris ΔKu_VVdCas9 Strains

A dCas9-HA fragment was amplified from pDTg1P_{GAP}dCas9 by PCR with a primer pair of HAPTg1UP F (SEQ ID NO: 99) and HAPTg1DO P (SEQ ID NO: 100). 100 ng, 3,5k-TEF1-gRNA1 plasmid and 1 g dCas9-HA fragment were transformed into Δku70 strain simultaneously, then the transformants were plated onto YND agar plates without histidine and cultured at 30° C. in an incubator for 48-72 hours. Single colonies grown on the plates were picked and transferred into liquid culture medium, then they were cultivated in an incubator shaker at 30° C. for genome extract ion. Verified transformants were streaked onto YPD plates, and single colonies grown on these plates were picked and transferred to liquid medium. Genome extraction was performed after cultivated in an incubator shaker at 30° C., and dCas9 copy number was validated using Real-time PCR. The Pichia pastoris strain with single-copy dCas9 expression was identified by Real-time PCR and designated as Δku_dCas9.

A recombinant plasmid pGP_{GAP}VRERVP16 was electroporated into Pichia pastoris strain Δku_dCas9, then the transformants were plated onto YPD agar plates supplemented with Zeocin antibiotic and cultured at 30° C. in an incubator for 48-72 hours. Single colonies grown on the plates were picked and transferred into liquid culture medium, cultivated in an incubator shaker at 30° C., then the genomic DNA was extracted, with the copy number of VP16 verified using Real-time PCR. The Pichia pastoris strain with single-copy VP16 expression was identified by Real-time PCR and designated as Δku_VVdCas9.

2. Construction of pPg2cATSAD Plasmid

A GFP encoding gene and the plasmid backbone region was amplified from pPAG by PCR with a primer pair of HP-GFP F (SEQ ID NO: 101) and HP-pP R (SEQ ID NO: 102). Subsequently, they were assembled with HP promoter using a seamless cloning kit to obtain the recombinant plasmid named pPHPGFP.

A AOX1 core promoter, a AOXTT terminator and the plasmid backbone region were amplified form the plasmid pPg2rcAG by PCR with a primer pair of STA-TT F (SEQ ID NO: 103) and STA-cA R (SEQ ID NO: 104). Subsequently, they were assembled with STA fragment using a seamless cloning kit to obtain the recombinant plasmid named pPg2rcASTA. In the STA (SEQ ID NO: 29), a region from position 1 to 1086 is LacI protein encoding sequence, wherein LacI can bind to corresponding operator sequence in HP; a region from position 1102 to 3456 is Mit1AD activation domain with a faction of transcriptional activation; wherein, the corresponding lac operator sequence in the 1HP (SEQ ID NO: 30) is at the position from 81 to 283 and can be recognized and bound by the LacI protein.

A HP promoter region, a GFP region and the plasmid backbone region were amplified from the plasmid pPHPGFP by PCR with a primer pair of TT-HP F (SEQ ID NO: 105) and inOri R (SEQ ID NO: 106); An AOX1 core promoter, a STA encoding gene and a AOXTT terminator region were amplified form pPg2rcASTA by PCR with a primer pair of inOri F (SEQ ID NO: 107) and HP-TT F (SEQ ID NO: 108). By assembling the two fragments using the Seamless Cloning Kit, a recombinant plasmid pPg2rcATSAD was obtained.

### 3. Screening of Pichia Pastoris ΔKu_VVdCas9-g2rcATSAD Strains

A recombinant plasmid pPg2rcATSAD was transformed into Pichia pastoris strain Δku_VVdCas9 by electroporation, then the transformants were plated onto YND agar plates lacking histidine and cultured at 30° C. in an incubator for 48-72 hours. Single colonies grown on the plates were picked and transferred into liquid culture medium, cultivated in an incubator shaker at 30° C., then the genomic DNA was extracted, with the copy number of GFP verified using Real-time PCR. The Pichia pastoris strain with single-copy GFP expression was identified by Real-time PCR and designated as Δku_VVdCas9-g2rcATSAD.

4. Construction of giRNA_1 and gaRNA_2 Co-Expressive Plasmid

The recombinant plasmid pAA-P_{GAP}gi1 was digested and linearized by XhoI/KpnI, with the longer fragments (˜5500 bp) recovered. Then it was assembled with amplified fragments of a AOX2 promoter, a ICL1 promoter, a GPM1 promoter, and a ENO1 promoter (with increasing promoter strengths under glucose conditions) obtained from the genome of P/chia pastoris strain GS115, using a seamless cloning kit to generate recombinant plasmids of pAA-P_{AOX2}gi1, pAA-P_{ICL1}gi1, pAA-P_{GPM1}gi1 and pAA-P_{ENO1}gi1, separately.
Primer pair for AOX2 promoter amplification: pAA-AOX2 F (SEQ ID NO: 109) and HH-AOX2 R (SEQ ID NO: 110);
Primer pair for ICL1 promoter amplification: pAA-CLI F (SEQ ID NO: 111) and HH-ICL1 R (SEQ ID NO: 112);
Primer pair for GPM1 promoter amplification: pAA-GPM1 F (SEQ ID NO: 113) and HH-GPM1 R (SEQ ID NO: 114);
Primer pair for ENO1 promoter amplification: pAA-ENO1 F (SEQ ID NO: 115) and HH-ENO1 R (SEQ ID NO: 116).

The recombinant plasmids pAA-P_{GAP}ga2 and pAA-P_{GAP}gi1 were digested and linearized separately by XhoI/KpnI, with the large fragment (˜5500 bp) and the short fragment (˜1000 bp) recovered, respectively. The two fragments were then ligated to obtain a recombinant plasmid of pAA-P_{AOX2}ga2. By similar methods, recombinant plasmids pAA-P_{ICL1}ga2, pAA-P_{GPM1}ga2 and pAA-P_{ENO1}ga2 were obtained.

The recombinant plasmid pAA-P_{GAP}ga2 was digested by XhoI/EcoRI, with the large fragment (˜5400 bp) recovered. The recombinant plasmids pAA-P_{ICL1}gi1, pAA-P_{ICL1}gi1, pAA-P_{ENO1}gi1, pAA-P_{GAP}gi1 were digested by EcoRI/SalI, with the short fragment recovered and ligated with the above fragments to generate recombinant plasmids pAA-P_{ICL1}gi1-P_{AOX2}ga2, pAA-P_{GPM1}gi1-P_{AOX2}ga2, pAA-P_{ENO1}gi1-P_{AOX2}ga2, pAA-P_{GAP}gi1-P_{AOX2}ga2. By similar methods, recombinant plasmids pAA-P_{AOX2}gi1-P_{ICL1}ga2, pAA-P_{GPM1}gi1-P_{ICL1}ga2, pAA-P_{ENO1}gi1-P_{ICL1}ga2, pAA-P_{GAP}gi1-P_{ICL1}ga2, pAA-P_{AOX2}gi1-P_{GPM1}ga2, pAA-P_{ICL1}gi1-P_{GPM1}ga2, pAA-P_{ENO1}gi1-P_{GPM1}ga2, pAA-P_{GAP}gi1-P_{ENO1}ga2, pAA-P_{AOX2}gi1-P_{ENO1}ga2, pAA-P_{ICL1}gi1-P_{ENO1}ga2, pAA-P_{GPM1}gi1-P_{ENO1}ga2, pAA-P_{GAP}gi1-P_{ENO1}ga2, pAA-P_{AOX2}gi1-P_{GAP}ga2, pAA-P_{ICL1}gi1-P_{GAP}ga2, pAA-P_{GPM1}gi1-P_{GAP}ga2 and pAA-P_{ENOL}gi1-P_{GAP}ga2 were obtained sequentially.

5. Screening of Electroporated Pichia pastoris and Single-Copy Strains

20 recombinant plasmids described above were electroporated into Pichia pastoris strain Δku_VVdCas9-g2rcATSAD, then the transformants were plated onto YPD agar plates supplemented with Hygromycin antibiotic and cultured at 30° C. in an incubator for 48-72 hours. Single colonies grown on the plates were picked and transferred into liquid culture medium, cultivated in an incubator shaker at 30° C., then the genomic DNA was extracted, with the copy number of giRNA_1 verified using Real-time PCR. Then a series of Pichia pastoris strains with single-copy giRNA_1 and gaRNA_2 expression by different promoters.

6. Detection of GFP Fluorescence Intensity by Microplate Reader The above 20 Pichia pastoris strains were separately pre-cultured overnight in YPD liquid medium, then the strains were collected by centrifugation, washed twice with distilled water and transferred to YNM liquid medium for cultivation. Samples were taken and GFP fluorescence intensity in the samples was detected using; a microplate reader.

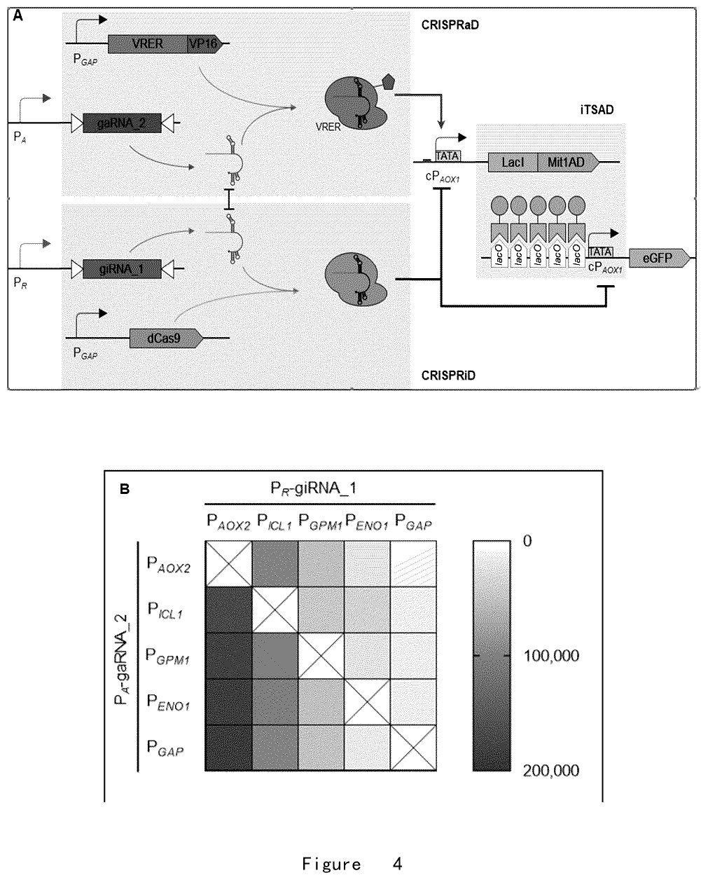

As shown in FIG. 4A-B, the results indicate that the output signal intensity of the artificial transcriptional regulation system mediated by VRER+gaRNA_2 decreases with increasing giRNA_1 expression level and increases with increasing gaRNA_2 expression level. When the expression level of giRNA_1 is at its highest (P_{GAP}) and the expression level of gaRNA_2 is at its lowest (P_{AOX2}), the overall system's output signal intensity is at its lowest, indicating a suppressed state: when the expression level of giRNA_1 is at its lowest (P_{AOX2}) and the expression level of gaRNA 2 is at its highest (P_{GAP}), the overall system's output intensity reaches its maximum level, indicating an activated state. The difference in output signal intensity can reach up to 29.1-fold, demonstrating excellent fine-tuning performance.

### Example 4. Interference Device (dCas9+giRNA_1) and Activation Device (dCpf1+craRNA_3) Mediated Artificial Transcription Regulatory Systems

In this example, the strain used is Pichia pastoris Δku70, and the main devices are as follows:

| | | Plasmids (main elements of the expression cassette) | Others |
|---|---|---|---|
| CRISPR | dCas9 | 3.5k-TEF1-gRNA1; dCas9-HA | |
| interference | giRNA_1 | (AOX2 promoter, ICL1 promoter, GPM1 promoter, | |
| devices | | ENO1 promoter, GAP promoter separately drives | |
| | | giRNA_1) | |
| CRISPR | dCpf1 | pGP_{GAP}dCpf1VP16 | VP16: Individually |
| activation | | (GAP promoter-dCpf1-VP16) | recruits RNA polymerase |
| devices | craRNA_3 | (AOX2 promoter, ICL1 promoter, GPM1 promoter, | |
| | | ENO1 promoter, GAP promoter separately drives | |
| | | craRNA_3) | |
| Effectors | GFP | pPcr3cATSAD (HP promoter-GFP and craRNA_3 | STA: signal |
| | | binding sequence-AOX1 core promoter-STA) | amplification |

Main Methods of Construction were as Follows:
1. Screening of Pichia pastoris Δku_dCVdCas9 Strains

A recombinant plasmid pGP_{GAP}dCpf1VP16 was electroporated into Pichia pastoris strain Δku_dCas9, then the transformants were plated onto YPD agar plates supplemented with Zeocin antibiotic and cultured at 30° C. in an incubator for 48-72 hours. Single colonies grown on the plates were picked and transferred into liquid culture medium, cultivated in an incubator shaker at 30° C., then the genomic DNA was extracted, with the copy number of VP16 verified using Real-time PCR. The Pichia pastoris strain with single-copy VP16 expression was identified by Real-time PCR and designated as Δku_dCVdCas9.

2. Construction of pPcr3cATSAD Plasmid

A AOX1 core promoter, a AOXTT terminator and the plasmid backbone region were amplified form the plasmid pPcr3cAG by PCR with a primer pair of STA-TT F (SEQ ID NO: 103) and STA-cA R (SEQ ID NO: 104). Subsequently, they were assembled with STA fragment using a seamless cloning kit to obtain the recombinant plasmid named pPcr3cASTA.

A HP promoter region, a GFP region and the plasmid backbone region were amplified from the plasmid pPHPGFP by PCR with a primer pair of TT-HP (SEQ ID NO: 105) and inOri R (SEQ ID NO: 106); An AOX1 core promoter, a STA encoding gene and a AOXTT terminator region were amplified form pPg2rcASTA by PCR with a primer pair of inOri F (SEQ ID NO: 107) and HP-TT (SEQ ID NO: 108). By assembling the two fragments using the Seamless Cloning Kit, a recombinant plasmid pPcr3cATSAD was obtained.

3. Screening of Pichia pastoris ΔKu_dCVdCas9-cr3cATSAD Strains

A recombinant plasmid pPcr3cATSAD was transformed into Pichia Pastoris strain Δku_dCVdCas9 by electroporation, then the transformants were plated onto YND agar plates lacking histidine and cultured at 30° C. in an incubator for 48-72 hours. Single colonies grown on the plates were picked and transferred into liquid culture medium, cultivated in an incubator shaker at 30° C., then the genomic DNA was extracted, with the copy number of GFP verified using Real-time PCR. The Pichia pastoris strain with single-copy GFP expression was identified by Real-time PCR and designated as Δku_dCVdCas9-cr3CATSAD.

4. Construction of giRNA_1 and craRNA_3 Co-Expressive Plasmid

The recombinant plasmids pAA-P_{GAP}cra3 and pAA-P_{AOX2}gi1 were digested and linearized separately by XhoI/KpnI, with the large fragment (˜5500 bp) and the short fragment (˜1000 bp) recovered, respectively. The two fragments were then ligated to obtain a recombinant plasmid of pAA-P_{AOX2}cra3. By similar methods, recombinant plasmids pAA-P_{ICL1}cra3, pAA-P_{GPM1}cra3 and pAA-P_{ENO1}cra3 were obtained.

The recombinant plasmid pAA-P_{ICL1}cra3 was digested by XhoI/EcoRI, with the large fragment (˜5400 bp) recovered. The recombinant plasmids pAA-P_{ICL1}gi1, pAA-P_{GPM1}gi1 pAA-P_{ENO1}gi1, pAA-P_{GAP}gi1 were digested by EcoRI/SalI, with the short fragment recovered and ligated with the above fragments to generate recombinant plasmids pAA-P_{ICL1}gi1-P_{AOX1}cra3, pAA-P_{GPM1}gi1-P_{AOX2}cra3, pAA-P_{ENO1}gi1-P_{AOX2}cra3, pAA-P_{GAP}gi1-P_{AOX2}cra3. By similar methods, recombinant plasmids pAA-P_{AOX2}gi1-P_{ICL1}cra3, pAA-P_{GPM1}gi1-P_{ICL1}cra3, pAA-P_{ENO1}gi1-P_{ICL1}cra3, pAA-P_{GAP}gi1-P_{ICL1}cra3, pAA-P_{AOX2}gi1-P_{GPM1}cra3, pAA-P_{ICL1}gi1-P_{GPM1}cra3, pAA-P_{ENO1}gi1-P_{GPM1}cra3, pAA-P_{GAP}gi1-P_{GPM1}cra3, pAA-P_{AOX2}gi1-P_{ENO1}cra3, pAA-P_{ICL1}gi1-P_{ENO1}cra3, pAA-P_{GPM1}gi1-P_{ENO1}cra3, pAA-P_{GAP}gi1-P_{ENO1}cra3, pAA-P_{AOX2}gi1-P_{GAP}cra3, pAA-P_{ICL1}gi1-P_{GAP}cra3, pAA-P_{GPM1}gi1-P_{GAP}cra3, pAA-P_{ENO1}gi1-P_{GAP}cra3 were obtained sequentially.

5. Screening of Electroporated Pichia pastoris and Single-Copy Strains

20 recombinant plasmids described above were electroporated into Pichia pastoris strain Δku_dCVdCas9-cr3cATSAD, then the transformants were plated onto YPD agar plates supplemented with Hygromycin antibiotic and cultured at 30° C. in an incubator for 48-72 hours. Single colonies grown on the plates were picked and transferred into liquid culture medium, cultivated in an incubator shaker at 30° C., then the genomic DNA was extracted, with the copy number of giRNA_1 verified using Real-time PCR.

Then a series of Pichia pastoris strains with single-copy giRNA_1 and craRNA3 expression by different promoters.

### 6. Detection of GFP Fluorescence Intensity by Microplate Reader

The above 20 Pichia pastoris strains were separately pre-cultured overnight in YPD liquid medium, then the strains were collected by centrifugation, washed twice with distilled water and transferred to YNM liquid medium for cultivation. Samples were taken and GFP fluorescence intensity in the samples was detected using a microplate reader.

As shown in FIG. 5A-B, the results indicate that the output signal intensity of the artificial transcriptional regulation system mediated by dCpf1+craRNA3 decreases with increasing giRNA_1 expression levels and increases with increasing craRNA_3 expression levels. When the expression level of giRNA_1 is at its highest (P_{GAP}) and the expression level of craRNA_3 is at its lowest (P_{AOX2}), the overall system's output signal intensity is at its lowest, indicating a suppressed state: when the expression level of giRNA_1 is at its lowest (P_{AOX2}) and the expression level of craRNA_3 is at its highest (P_{GAP}) the overall system's output intensity reaches its maximum level, indicating an activated state. The difference in output signal intensity can reach up to 23.4-fold. Compared to the artificial transcriptional regulation system mediated by VRER+gaRNA_2, this system exhibits stricter regulation, a wider range of signal modulation, and is more suitable for fine-tuning gene expression.

### Example 5. Development of Rhamnose-Suppressing Expression System

In this example, the strain used is Pichia Pastoris AK‘u’0, and the main devices are as follows:

| | | Plasmids (main elements of the expression cassette) | Others |
|---|---|---|---|
| CRISPR | dCas9 | GAP promoter-dCas9 | |
| interference | giRNA_1 | LRA3 promoter drives giRNA_1 | LRA3: rhamnose-inducible |
| devices | | | promoter |
| CRISPR | kCpf1 | pGP_{GAP}dCpf1VP16 (GAP promoter-dCpf1-VP16) | VP16: Individually recruits |
| activation | | | RNA polymerase |
| devices | craRNA_3 | GAP promotor drives craRNA_3 | |
| Effectors | GFP | pPcr3cATSAD (HP promoter-GFP and craRNA_3 | STA: signal amplification |
| | | binding sequence-AOX1 core promoter-STA) | |

Main Methods of Construction were as Follows:
1. Construction of pAA-P_{LRA3}Gi1-P_{GAP}Cra3 Plasmid

The recombinant plasmid pAA-P_{GAP}gi1 was digested and linearized separately by XhoI/KpnI, with the large fragment (˜5500 bp) recovered. A fragment of LRA3 promoter was amplified from Pichia pastoris GS115 genome by PCR with a primer pair of pAA-LRA3 Y (SEQ ID NO: 117) and HH-LRA3 R (SEQ ID NO: 118). By assembling the two fragments using the Seamless Cloning Kit, a recombinant plasmid pAA-P_{LRA3}gi1 was obtained.

A recombinant plasmid pAA-P_{GAP}cra3 was digested by XhoI/MluI, with the large fragment (˜5700 bp) recovered; A recombinant plasmid pAA-P_{LRA3}gi1 was digested by MluI/SalI, with the short fragment (˜1000 bp) recovered. By ligating the two fragments, a recombinant plasmid pAA-P_{LRA3}gi1-P_{GAP}cra3 was obtained

2. Screening of Pichia pastoris ΔKu_dCVdCas9-cr3cATSAD-LRA3gi1GAPcra3 Strains

A recombinant plasmid pAA-P_{LRA3}gi1-P_{GAP}cra3 was electroporated into Pichia pastoris strain Δku_dCVdCas9-cr3cATSAD, then the transformants were plated onto YPD agar plates supplemented with Hygromycin antibiotic and cultured at 30° C. in an incubator for 48-72 hours. Single colonies grown on the plates were picked and transferred into liquid culture medium, cultivated in an incubator shaker at 30° C., then the genomic DNA was extracted, with the copy number of giRNA_1 verified using Real-time PCR. The Pichia pastoris strain with single-copy giRNA_1 expression was identified by Real-time PCR and designated as Δku_dCVdCas9-cr3cATSAD-LRA3gi1GAPcra3.

3. Detection of GFP Fluorescence Intensity with Various Carbon Sources

The strain Δku_dCVdCas9-cr3cATSAD-LRA3gi1GAPcra3 was pre-cultured overnight in YPD liquid medium, then the strains were collected by centrifugation, washed twice with distilled water and transferred to a liquid medium containing glucose (YPD), glycerol (YPG), ethanol (YNE), methanol (YNM) and raffinose (YPR) for cultivation. Samples were taken and GFP fluorescence intensity in the samples was detected using a microplate reader.

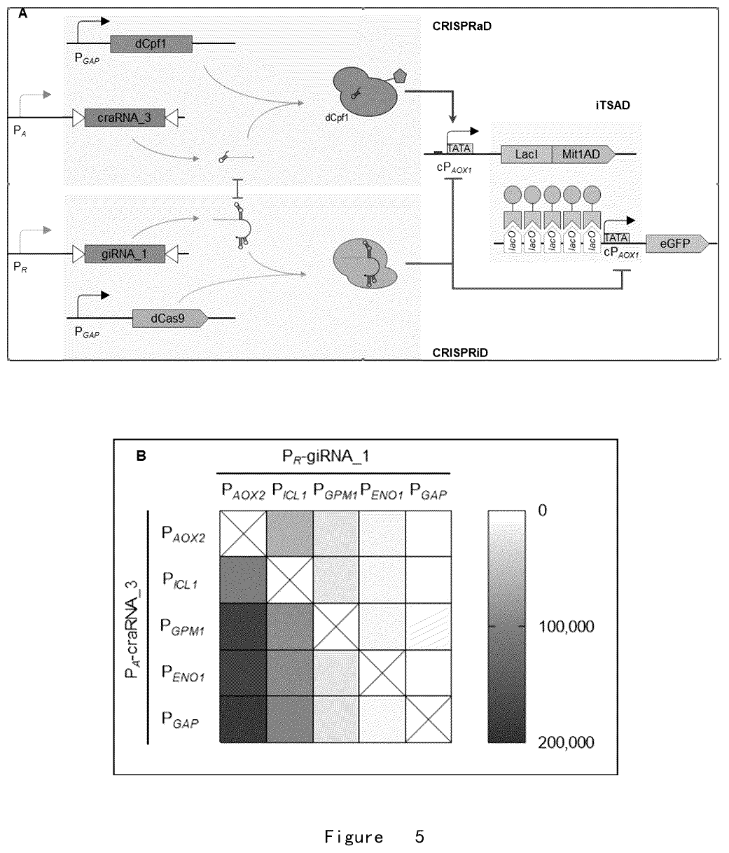

As shown in FIG. 6, when raffinose serves as a carbon source, the LRA3 promoter is activated, leading to an increased expression of giRNA_1 and a suppression of the expression system, putting it in the “Off” state. When glucose, glycerol, ethanol, methanol, or other substance serves as carbon sources, the LRA3 promoter is suppressed, resulting in a suppression of giRNA_1 expression and activation of the expression system (the artificial transcriptional regulation system mediated by the activation devices dCpf1+craRNA3 is running), putting it in the “On” state. Wherein, the rhamnose-suppressing expression system shows the highest output intensity under conditions with glucose, with a difference of 22.9-fold eGFP expression levels compared to the “Off” state under conditions with raffinose. This system achieves efficient expression and precise regulation, demonstrating great potential for practical applications.

4. Detection of GFP Fluorescence Intensity with Various Rhamnose Concentrations

The strain Δku_dCVdCas9-cr3cATSAD-LRA3gi1GAPcra3 was pre-cultured overnight in a YPD liquid medium, then the strain was collected by centrifugation, washed twice with distilled water and transferred to a series of YP liquid medium including different concentrations of raffinose (with 20, 15, 10, 5, 2.5, 1, 0.5, 0.25, 0.2, 0.08, 0.025, 0.016, 0.01, 0.0064, 0.0025, 0.00128, 0.001, 0.000512, 0.00025, 0.0001, 0.000025, 0.00001, 0.0000025 g/L, respectively) for cultivation. Samples were taken, and the fluorescence intensity of GP in the samples was measured using a microplate reader.

As shown in FIG. 7, the output signal of this expression system exhibits a clear dose-response relationship with raffinose concentration. The intensity of the output signal increases as the raffinose concentration decreases. When the raffinose concentration is below 0.0001 g/L, the output intensity essentially reaches its maximum level, indicating an “On” state. The difference in output intensity of this expression system under different raffinose concentrations can reach up to 24,1-fold, demonstrating an excellent fine-tuning performance.

In this example, the activation devices are all driven by the GAP promoter, a constitutively expressive promoter. After the interference devices are inhibited, the system turns to an activated state.

### Example 6. Development of Methanol-Suppressing Expression System

In this example, the strain used is Pichia pastoris Δku70, and the main devices are as follows.

| | | Plasmids (main elements of the expression cassette) | Others |
|---|---|---|---|
| CRISPR | dCas9 | GAP promoter-dCas9 | |
| interference | giRNA_1 | DAS1 promoter drives giRNA_1 | DAS1: Methanol inducible |
| devices | | | promoter |
| CRISPR | dCpf1 | pGP_{GAP}dCpf1VP16 (GAP promoter-dCpf1-VP16) | VP16: Individually recruits |
| activation | | | RNA polymerase |
| devices | craRNA_3 | GAP promoter drives craRNA_3 | |
| Effectors | GFP | pPcr3cATSAD (HP promoter-GFP and | STA; signal amplification |
| | | craRNA_3 binding sequence-AOX1 core | |
| | | promoter-STA) | |

Main Methods of Construction were as Follows:
1. Construction of pAA-P_{DAS1}Gi1-P_{GAP}cra3 Plasmid

The recombinant plasmid pAA-P_{GAP}gi1 was digested and linearized by XhoI/KpnI, with the large fragment (˜5500 bp) recovered. A fragment of DAS1 promoter was amplified from Pichia pastoris GS115 genome by PCR with a primer pair of pAA-DAS1 F (SEQ ID NO: 119) and HH-DAS1 R (SEQ ID NO: 120). By assembling the two fragments using the Seamless Cloning Kit, a recombinant plasmid pAA-P_{DAS1}gi1 was obtained.

A recombinant plasmid pAA-P_{GAP}cra3 was digested by XhoI/MluI, with the large fragment bp) recovered; A recombinant plasmid pAA-P_{DAS1}gi1 was digested by MluI/SalI, with the short fragment (˜1800 bp) recovered. By ligating the two fragments, a recombinant plasmid pAA-P_{DAS1}gi1-P_{GAP}cra3 was obtained

2. Screening of Pichia pastoris ΔKu_dCVdCas9-cr3cATSAD-DAS1gi1GAPcra3 Strains

A recombinant plasmid pAA-P_{DAS1}gi1-P_{GAP}cra3 was electroporated into Pichia pastoris strain Δku_dCVdCas9-cr3cATSAD, then the transformants were plated onto YPD agar plates supplemented with Hygromycin antibiotic and cultured at 30° C. in an incubator for 48-72 hours. Single colonies grown on the plates were picked and transferred into liquid culture medium, cultivated in an incubator shaker at 30° C., then the genomic DNA was extracted, with the copy number of giRNA-1 verified using Real-time PCR.

The Pichia pastoris strain with single-copy giRNA_1 expression was identified by Real-time PCR and designated as Δku_dCVdCas9-cr3cATSAD-DAS1gi1GAPcra3.

3. Detection of GFP Fluorescence Intensity with Various Carbon Sources

The strain Δku_dCVdCas9-cr3cATSAD-DAS1gi1GAPcra3 was pre-cultured overnight in YPD liquid medium, then the strains were collected by centrifugation, washed twice with distilled water and transferred to a liquid medium containing glucose (YPD), glycerol (YPG), ethanol (YNE) and methanol (YNM) for cultivation. Samples were taken and GFP fluorescence intensity in the samples was detected using a microplate reader.

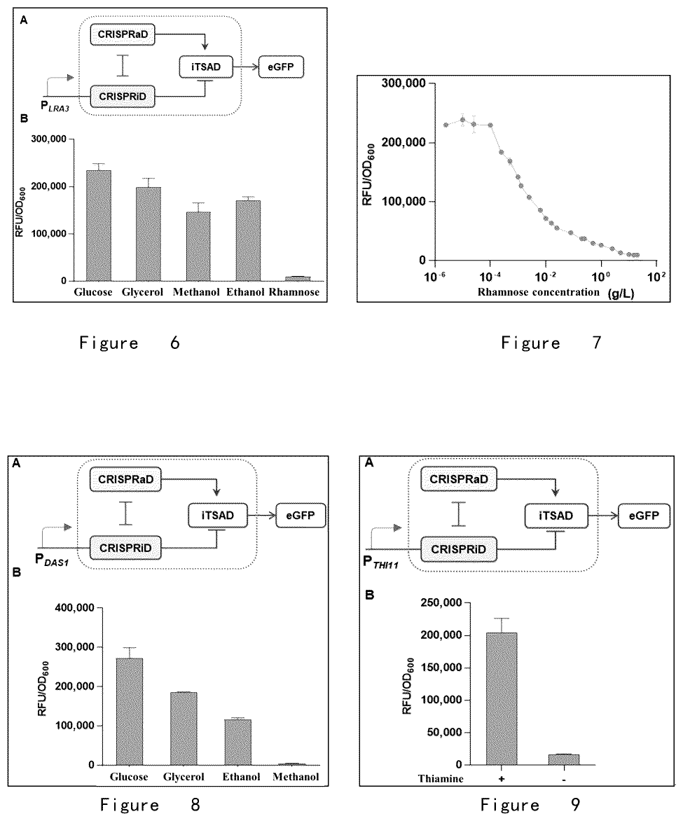

As shown in FIG. 8, the results indicate that under methanol conditions, the DAS1 promoter is activated, leading to an increased expression of giRNA_1 and a suppression of the expression system, putting it in the “Off” state. When glucose, glycerol, ethanol or other substance serves as carbon sources, the DAS1 promoter is suppressed, resulting in a suppression of giRNA_1 expression and activation of the expression system, putting it in the “On” state. Wherein, the methanol-suppressing expression system shows the highest output intensity under conditions with glucose, with a difference of 54.3-fold eGFP expression levels compared to the “Off” state under conditions with methanol. This system achieves efficient expression and precise regulation, demonstrating great potential for practical applications.

In this example, the activation devices are all driven by the GAP promoter, a constitutively expressive promoter. After the interference devices are inhibited, the system turns to an activated state.

### Example 7. Development of Thiamine Inducible Expression System

In this example, the strain used is Pichia pastoris Δku70, and the main devices are as follows:

| | | Plasmids (main elements of the | |
|---|---|---|---|
| | | expression cassette) | Others |
| CRISPR | dCas9 | GAP promoter-dCas9 | |
| interference | giRNA_1 | THI11 promoter drives giRNA_1 | THI11: A promoter induced by |
| devices | | | thiamine starvation |
| CRISPR | Cpf1 | pGP_{GAP}dCpf1VP16 | VP16: Individually recruits RNA |
| activation | | (GAP promoter-dCpf1-VP16) | polymerase |
| devices | craRNA_3 | GAP promoter drives craRNA_3 | |
| Effectors | GFP | pPcr3cATSAD | STA: signal amplification |
| | | (HP promoter-GFP and craRNA_3 | |
| | | binding sequence-AOX1 core | |
| | | promoter-STA) | |

Main Methods of Construction were as Follows:
1. Construction of pAA-P_{THI11}Gi1-P_{GAP}Cra3 Plasmid

A recombinant plasmid pAA-P_{GAP}gi1 was digested and linearized separately by XhoI/KpnI, with the large fragment (˜5500 bp) recovered. A fragment of THI11 promoter was amplified from Pichia pastoris GS115 genome by PCR with a primer pair of pAA-THI11 F (SEQ ID NO: 121) and HH-THI11 R (SEQ ID NO: 122). By assembling the two fragments using the Seamless Cloning Kit, a recombinant plasmid pAA-P_{THI11}gi1 was obtained.

A recombinant plasmid pAA-P_{GAP}cra3 was digested by XhoI/MluI, with the large fragment (˜5700 bp) recovered; A recombinant plasmid pAA-P_{THI11}gi1 was digested by MluI/SalI, with the short fragment (˜1800 bp) recovered. By ligating the two fragments, a recombinant plasmid pAA-P_{THI11}gi1-P_{GAP}cra3 was obtained.

2. Screening of Pichia pastoris ΔKu_dCVdCas9-cr3cATSAD-TH111gi1GAPcra3 Strains

A recombinant plasmid pAA-P_{THI11}gi1-P_{GAP}cra3 was electroporated into Pichia pastoris strain Δku_dCVdCas9-cr3cATSAD, then the transformants were plated onto YPD agar plates supplemented with Hygromycin antibiotic and cultured at 30° C. in an incubator for 48-72 hours. Single colonies grown on the plates were picked and transferred into liquid culture medium, cultivated in an incubator shaker at 30° C., then the genomic DNA was extracted, with the copy number of giRNA_1 verified using Real-time PCR. The Pichia pastoris strain with single-copy giRNA_1 expression was identified by Real-time PCR and designated as Δku_dCVdCas9-cr3cATSAD-TH111gi1GAPcra3.

### 3. Detection of GFP Fluorescence Intensity Under Various Conditions

The strain Δku_dCVdCas9-cr3cATSAD-THI11gi1GAPcra3 was pre-cultured overnight in YPD liquid medium, then the strains were collected by centrifugation, washed twice, with distilled water and transferred to a synthetic medium containing 0 or 4 mmol/L thiamine for cultivation. Samples were taken and GFP fluorescence intensity in the samples was detected using a microplate reader.

As shown in FIG. 9, the results indicate that under no thiamine conditions, the THI11 promoter is activated, leading to an increased expression of giRNA_1 and a suppression of the expression system, putting it in the “Off” state. Under thiamine conditions, the THI11 promoter is suppressed, resulting in a suppression of giRNA_1 expression and activation of the expression system, putting it in the “On” state. The thiamine-inducible expression system shows a difference of 12.5-fold eGFP expression levels, indicating great regulatory functions.

Each reference provided herein is incorporated by reference to the same extent as if each reference was individually incorporated by reference. In addition, it should be understood that based on the above teaching content of the disclosure, those skilled in the art can practice various changes or modifications to the disclosure, and these equivalent forms also fall within the scope of the appended claims.

1-17. (canceled)

18. A transcription regulation system based on CRISPRi and CRISPRa, comprising:
a signaling effector device comprising a target promoter and a target gene operably linked thereto;
a CRISPR interference (CRISPRi) device targeting and interfering the target promoter and decreasing the expression of the target gene driven by the target promoter;
a CRISPR activation (CRISPRa) device targeting and activating the target promoter and increasing the expression of the target gene driven by the target promoter.

19. The transcription regulation system according to claim 18, wherein the CRISPR interference device comprises: an expression cassette a, expressing the inactivated Cas protein 1 based on the CRISPR system; and, an expression cassette b, expressing a guide RNA, namely giRNA, the giRNA guides the inactivated Cas protein 1 to the target promoter region in the signaling effector device;
the CRISPR activation device comprises: an expression cassette c, expressing a fused peptide of the inactivated Cas protein 2 based on the CRISPR system and a transcriptional activator; and, an expression cassette d, expressing a guide RNA, namely gaRNA or craRNA, and the gaRNA or craRNA guides the inactivated Cas protein 2 to the target promoter region in the signaling effector device;
wherein, the inactivated Cas protein 1 and the inactivated Cas protein 2 recognize different PAM sequences in the target promoter sequence and are orthogonal to each other; the giRNA and gaRNA or craRNA can form giRNA-gaRNA dimer or giRNA-craRNA dimer, and interact with each other to regulate the strength of interference or activation; preferably, the gaRNA or craRNA is complementary to the partial sequence of the giRNA to form a dimer.

20. The transcription regulation system according to claim 19, wherein the giRNA comprises a segment a and a Cas protein binding region a, and the segment a is complementary to the target promoter in the signaling effector device; the gaRNA or the craRNA comprises a segment b and a Cas protein binding region b; the segment b is complementary to segment a, or the segment a or b is complementary to the Cas protein binding region a or b.

21. The transcription regulation system according to claim 19, wherein, the expression cassette a comprises a promoter driving the expression of the inactivated Cas protein 1; preferably, the promoter comprises: a constitutive promoter or an inducible promoter; more preferably, the promoter comprises: a GAP promoter, a ENO1 promoter, a GPM1 promoter, a ICL1 promoter, a AOX2 promoter, a TEF1 promoter, a PGK1 promoter, a GTH1 promoter, a DAS1 promoter, a FBA2 promoter, a THI11 promoter, a LRA3 promoter; preferably, the promoter in the expression cassette a is different from the target promoter in the signaling effector device.

22. The transcription regulation system according to claim 19, wherein, in the expression cassette a, the inactivated Cas protein 1 is a Cas protein or a mutant thereof without nuclease activity; preferably, it is dCas9; preferably, the nucleotide sequence of the dCas9 gene is a sequence as shown in SEQ ID NO: 1 or a degenerate sequence thereof.

23. The transcription regulation system according to claim 19, wherein, the expression cassette b comprises a promoter driving the expression of giRNA; preferably, the promoter comprises: a constitutive promoter or an inducible promoter; preferably, the constitutive promoter comprises: a GAP promoter, a ENO1 promoter, a GPM1 promoter, a TEF1 promoter, a PGK1 promoter; preferably, the inducible promoter comprises: a rhamnose-inducible promoter, a methanol-inducible promoter, a thiamine-starvation-inducible promoter; more preferably, the rhamnose-inducible promoter comprises a LRA3 promoter, and the methanol-inducible promoter comprises a DAS1 promoter, a FBA2 promoter, or the thiamine starvation-inducible promoter comprises a THI11 promoter; preferably, the promoter in the expression cassette b is different from the target promoter in the signaling effector device.

24. The transcription regulation system according to claim 19, wherein, in the expression cassette b, the giRNA guides the inactivated Cas protein 1 in the expression cassette a to the target promoter region in the signaling effector device.

25. The transcription regulation system according to claim 19, wherein, the expression cassette c comprises a promoter driving the expression of the fused peptide with the inactivated Cas protein 2 and the transcriptional activator; preferably, the promoter comprises: a constitutive promoter or an inducible promoter; more preferably, the promoter comprises: a GAP promoter, a ENO1 promoter, a GPM1 promoter, a ICL1 promoter, a AOX2 promoter, a TEF1 promoter, a PGK1 promoter, a GTH1 promoter, a DAS1 promoter, a FBA2 promoter, a THI11 promoter, a LRA3 promoter; preferably, the promoter in the expression cassette c is different from the target promoter in the signaling effector device.

26. The transcription regulation system according to claim 19, wherein, in the expression cassette c, the inactivated Cas protein 2 is a Cas protein or a mutant thereof without nuclease activity; preferably, it comprises VRER or dCpf1; preferably, the nucleotide sequence of the VEGF gene is a sequence as shown in SEQ ID NO: 7 or a degenerate sequence thereof, the nucleotide sequence of the dCpf1 gene is a sequence as shown in SEQ ID NO: 8 or a degenerate sequence thereof.

27. The transcription regulation system according to claim 19, wherein, the expression cassette d comprises a promoter driving the expression of gaRNA or craRNA; preferably, the promoter comprises: a constitutive promoter or an inducible promoter; preferably, the constitutive promoter comprises: a GAP promoter, a ENO1 promoter, a GPM1 promoter, a TEF1 promoter, a PGK1 promoter; preferably, the inducible promoter comprises: a rhamnose-inducible promoter, a methanol-inducible promoter, a thiamine-starvation-inducible promoter; more preferably, the rhamnose-inducible promoter comprises a LRA3 promoter, and the methanol-inducible promoter comprises a DAS1 promoter, a FBA2 promoter, or the thiamine starvation-inducible promoter comprises a THI11 promoter; preferably, the promoter in the expression cassette d is different from the target promoter in the signaling effector device.

28. The transcription regulation system according to claim 19, wherein, in the expression cassette d, the gaRNA or craRNA guides the inactivated Cas protein 2 in the expression cassette c to the target promoter region in the signaling effector device.

29. The transcription regulation system according to claim 19, wherein, the transcriptional activator is a transcription factor with the ability to recruit RNA polymerase independently; preferably, it is VP16, VP64 or VPR; preferably, the nucleotide sequence of the VP16 gene is a sequence as shown in SEQ ID NO: 9 or a degenerate sequence thereof.

30. The transcription regulation system according to claim 19, wherein, the length of the giRNA is 50-300 bases; preferably the segment a is located at the 5′-terminal of the giRNA, more preferably the length of segment a is 10-50 bases; preferably, the segment b is located at the 5′-terminal of the gaRNA or the 3′ end of the craRNA, and its length is corresponding to the segment a;
the Cas protein binding region a or the Cas protein binding region b has at least 1 stem-loop in the secondary structure.

31. The transcription regulation system according to claim 19, wherein, the target promoter comprises a core promoter, the core promoter is a minimal promoter region with basic transcriptional activity; preferably, the target promoter comprises: a AOX1 promoter or a AOX1 core promoter; more preferably, the sequence of the AOX1 core promoter is shown in SEQ ID NO: 28.

32. The transcription regulation system according to claim 27, wherein, the target promoter is a AOX1 promoter or a AOX1 core promoter; the DNA sequence corresponding to the giRNA is shown in any one of SEQ ID NO: 2-6; or
the DNA sequence corresponding to the segment a is shown at the 1st to 21st positions in SEQ ID NO: 2, the 1st to 20th positions in SEQ ID NO: 3 or the 1st to 20th positions in SEQ ID NO: 4; or
the DNA sequence corresponding to the Cas protein binding region a is shown at the 22nd to 101st positions in SEQ ID NO: 2 or the 22nd to 101st positions in SEQ ID NO: 6.

33. The transcription regulation system according to claim 27, wherein, the RNA sequence corresponding to the gaRNA is shown in any one of SEQ ID NO: 10-12, preferably shown in SEQ ID NO: 11; the RNA sequence corresponding to the craRNA is shown in any one of SEQ ID NO: 13-15, preferably shown in SEQ ID NO: 15; or
the DNA sequence corresponding to the segment b is shown at the 1st to the 21st positions of SEQ ID NO: 10, the 1st to the 21st positions of SEQ ID NO: 11 or the 1st to the 91st positions of SEQ ID NO: 12; or shown at the 21st to 40th positions in SEQ ID NO: 13, the 21st to 42nd positions in SEQ ID NO: 14 or the 21st to 40th positions in SEQ ID NO: 15; or
the DNA sequence corresponding to the Cas protein binding region b is shown at the 22nd to 101st positions in SEQ ID NO: 10 or the 22nd to 101st positions in SEQ ID NO: 11; or shown at the 1st to 20th positions in SEQ ID NO: 13.

34. The transcription regulation system according to claim 18, wherein, the signaling effector device comprises sequentially operatively linked from 5′ to 3′: a gaRNA-binding sequence or a craRNA-binding sequence, a target promoter and a target gene; preferably, the gaRNA-binding sequence or the craRNA-binding sequence can bind to the corresponding gaRNA or craRNA with a template strand or a non-template strand; wherein, the gaRNA-binding sequence is shown in any one of SEQ ID NO: 16-21; the craRNA binding sequence is shown in any one of SEQ ID NO: 22-27; or
the signaling effector device also comprises a signal amplification element and an intermediate promoter activated thereby; preferably, the signal effect device comprises: (a) a target promoter and a signal amplification element driven thereby; and (b) an intermediate promoter that can be activated by the signal amplification element and the target gene expressed thereby; more preferably, the signal strengthen device comprises an artificial transcription activator STA, a hybrid promoter HP and an HP-driven target gene.

35. A method for regulating the expression of a target gene, wherein the method comprises: establishing a transcriptional regulation system according to any one of claim 18, and interfering or activating the expression according to the expected value of expression intensity of the target gene.

36. The method according to claim 32, wherein, the CRISPR interference device comprises a giRNA as a guide RNA and a dCas9 as an inactivated Cas protein 1; the CRISPR activation device comprises a gaRNA as a guide RNA and a VRER as an inactivated Cas protein 2; when the giRNA and gaRNA are expressed in different intensities, the target gene is expressed in different intensities; or
the CRISPR interference device comprises a giRNA as a guide RNA and a dCas9 as an inactivated Cas protein 1; the CRISPR activation device comprises a craRNA as a guide RNA and a dCpf1 as an inactivated Cas protein 2; when the giRNA and craRNA are expressed in different intensities, the target gene is expressed in different intensities; or
the CRISPR interference device comprises a giRNA as a guide RNA, with an inducible promoter used for regulating the expression of giRNA, and a dCas9 as an inactive Cas protein 1; the CRISPR activation device comprises a craRNA as a guide RNA, and a dCpf1 as an inactive Cas protein 2; when giRNA and craRNA are expressed in different intensities, the target gene is expressed in different intensities; preferably, the inducible promoters comprises: a rhamnose-inducible promoter, a methanol-inducible promoters, a thiamine-starvation inducible promoter.

37. A kit for regulating the expression of a target gene, wherein it comprises the transcription regulation system according to claim 18.


SEQUENCE LISTING (122 sequences)

SEQ ID NO: 1 - DNA, 4119 bp, synthetic construct
  source 1..4119: mol_type = other DNA
atggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgactaa

SEQ ID NO: 2 - DNA, 101 bp, synthetic construct
  source 1..101: mol_type = other DNA
tgacagcaatatataaacagagttttagagctagaaatagcaagttaaaataaggctagtccgttatcaacttgaaaaagtggcaccgagtcggtgctttt

SEQ ID NO: 3 - DNA, 100 bp, synthetic construct
  source 1..100: mol_type = other DNA
tttatatattgctgtcaagtgttttagagctagaaatagcaagttaaaataaggctagtccgttatcaacttgaaaaagtggcaccgagtcggtgctttt

SEQ ID NO: 4 - DNA, 100 bp, synthetic construct
  source 1..100: mol_type = other DNA
aataatgatgataaaaaaaagttttagagctagaaatagcaagttaaaataaggctagtccgttatcaacttgaaaaagtggcaccgagtcggtgctttt

SEQ ID NO: 5 - DNA, 151 bp, synthetic construct
  source 1..151: mol_type = other DNA
gatacttttcagagagcaatatatattgggttatatcttgctctcagaaatgacagcaatatataaacagagttttagagctagaaatagcaagttaaaataaggctagtccgttatcaacttgaaaaagtggcaccgagtcggtgctttt

SEQ ID NO: 6 - DNA, 101 bp, synthetic construct
  source 1..101: mol_type = other DNA
tgacagcaatatataaacagagttttagagctagaaatagcaagttaaaataaggctagtccgttatcaacttgaaaaagtgtctgctagtagcagatatt

SEQ ID NO: 7 - DNA, 4116 bp, synthetic construct
  source 1..4116: mol_type = other DNA
atggacaagaagtactccattgggctcgctatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgtttctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgcgcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaaggagtacaggtctacaaaggaggtcctggacgccacactgattcatcagtcaattacggggctctatgaaacaagaatcgacctctctcagctcggtggagacagcagggctgac

SEQ ID NO: 8 - DNA, 3684 bp, synthetic construct
  source 1..3684: mol_type = other DNA
atgagcaagctggagaagttcaccaactgctacagcctgagcaagaccctgagattcaaggccatccccgtgggaaaaacccaggagaacatcgacaacaagagactgctggtggaggacgaaaagagagccgaggactacaagggcgtgaagaagctgctggacagatactacctgagcttcatcaacgacgtgctgcacagcatcaagctgaagaacctgaacaactacatcagcctgttcagaaagaagaccagaaccgagaaggagaacaaggagctggagaacctggagatcaacctgagaaaggagatcgccaaggccttcaagggaaacgagggctacaagagcctgttcaagaaggacatcatcgagaccatcctgcccgagttcctggatgacaaggacgagatcgccctggtgaacagcttcaacggcttcaccaccgctttcaccggcttcttcgacaacagagagaacatgttcagcgaggaggccaagtctacaagcatcgccttcagatgcatcaacgagaacctgaccagatacatcagcaacatggacatcttcgagaaggtggacgccatcttcgacaagcacgaggtgcaggagatcaaggagaagatcctgaacagcgactacgacgtggaggacttcttcgagggcgagttcttcaacttcgtgctgacccaggaaggcatcgacgtgtacaacgccatcatcggcggatttgtgacagagagcggcgagaaaatcaagggcctgaacgagtacatcaacctgtacaaccagaagaccaagcagaagctgcccaagttcaagcccctgtacaagcaggtgctgagcgacagagagagcctgagcttctatggcgagggctacaccagcgatgaagaggtgctggaggtgttcagaaacaccctgaacaagaacagcgagatcttcagcagcatcaagaagctggagaagctgttcaagaacttcgacgagtacagcagcgccggcatctttgtgaaaaacggccccgctatcagcacaatcagcaaggacatcttcggcgagtggaacgtgatcagagacaagtggaacgccgagtacgacgacatccacctgaagaagaaggccgtggtgaccgagaaatacgaggacgacagaagaaagagcttcaagaagatcggcagcttcagcctggaacagctgcaagagtacgctgacgctgacctgagcgttgtggagaagctgaaggagatcatcatccagaaggtggacgagatctacaaggtgtacggcagcagcgagaaacttttcgacgccgacttcgtgcttgagaagagcctgaagaagaacgatgccgtggtggccatcatgaaggacctgctggacagcgtgaagagcttcgagaactacatcaaggccttcttcggcgaaggcaaggagaccaacagagacgagagcttctacggcgacttcgtgctggcttacgacatcctgctgaaggtggaccacatctacgacgccatcagaaactacgtgacccagaagccctacagcaaggacaagttcaagctgtacttccagaacccccagtttatgggcggatgggacaaggataaggagaccgactacagagccaccatcctgagatacggcagcaagtactacctggccatcatggacaagaagtacgccaagtgcctgcagaagatcgacaaggacgacgtgaacggcaactacgagaagatcaactacaagctgctgcccggccctaataaaatgctgcccaaggtgttcttcagcaagaagtggatggcctactacaaccccagcgaggacatccagaagatctacaagaacggcaccttcaagaagggcgacatgttcaacctgaacgactgccacaagctgatcgacttcttcaaggacagcatcagcagataccccaagtggagcaacgcctacgacttcaacttcagcgagaccgagaagtacaaggacatcgccggcttctacagagaagtggaggagcagggatacaaggtgagcttcgagagcgccagcaagaaggaggtggacaagctggtggaagagggcaagctgtacatgttccagatctacaacaaggacttcagcgacaagtctcacggaacccccaatctgcacaccatgtacttcaagctgctgttcgacgagaacaaccacggccagatcagactttctggaggcgctgaactgttcatgagaagagccagcctgaagaaggaagagctggtggtgcatcctgccaatagccccatcgctaacaagaaccccgacaaccccaagaaaaccaccaccctgagctacgacgtgtacaaggacaagagattcagcgaggaccagtacgagctgcatatccccatcgccatcaacaagtgccccaagaacatcttcaagatcaacaccgaggtgagagtgctgctgaagcacgacgacaacccctacgtgatcggcattgccagaggcgagagaaacctgctgtacatcgtggtggtggacggcaagggaaacatcgtggagcagtacagcctgaacgagatcatcaacaacttcaacggcatcagaatcaagaccgactaccacagcctgctggacaagaaggagaaggagagattcgaggccagacagaactggaccagcatcgagaacatcaaggagctgaaggccggctacattagccaggtggtgcacaagatctgcgagctggtggagaagtacgatgccgtgatcgctctggaggatctgaacagcggcttcaagaacagcagagtgaaggtggagaagcaggtgtaccagaagttcgagaagatgctgatcgacaagctgaactacatggtggacaagaagagcaacccctgtgctacaggcggagctctgaagggataccagatcaccaacaagttcgagagcttcaagagcatgagcacccagaacggcttcatcttctacatccccgcctggctgacatctaagatcgaccctagcaccggctttgtgaacctgctgaagaccaagtacaccagcatcgccgacagcaagaagttcatcagcagcttcgacagaatcatgtacgtgcccgaggaggacctgtttgaatttgccctggactacaagaacttcagcagaaccgacgccgactacatcaagaagtggaagctgtacagctacggcaacagaatcagaatcttcagaaaccccaagaagaacaacgtgttcgactgggaggaggtgtgtctgacaagcgcctacaaggagctgttcaacaagtacggcatcaactaccagcagggcgacattagagccctgctgtgcgaacagagcgacaaggccttctacagcagcttcatggccctgatgagcctgatgctgcagatgagaaacagcatcaccggcagaaccgacgtggacttccttatcagccccgtgaaaaacagcgacggcatcttctacgacagcagaaactacgaggcccaggagaatgctatcctgcccaagaatgccgatgctaacggcgcttacaacatcgccagaaaggtgctttgggccatcggccagtttaagaaggccgaggacgagaagctggacaaggtgaagatcgccatcagcaacaaggagtggctggagtatgctcagaccagcgtgaaacac

SEQ ID NO: 9 - DNA, 237 bp, synthetic construct
  source 1..237: mol_type = other DNA
gctccaccaaccgacgtttctttgggtgacgagttgcacttggacggtgaagatgttgccatggctcatgctgacgctttggacgacttcgacttggacatgttgggtgacggtgattctccaggtccaggtttcactccacacgattctgctccatacggtgctttggacatggccgacttcgagtttgagcagatgttcaccgacgctttgggtattgacgagtacggtggttaa

SEQ ID NO: 10 - DNA, 101 bp, synthetic construct
  source 1..101: mol_type = other DNA
tctgtttatatattgctgtcagttttagagctagaaatagcaagttaaaataaggctagtccgttatcaacttgaaaaagtggcaccgagtcggtgctttt

SEQ ID NO: 11 - DNA, 101 bp, synthetic construct
  source 1..101: mol_type = other DNA
tagctcttaaagtctgtttatgttttagagtcagaaatgacaagttaaaataaggctagtccgttatcaacttgaaaaagtggcaccgagtcggtgctttt

SEQ ID NO: 12 - DNA, 171 bp, synthetic construct
  source 1..171: mol_type = other DNA
cgtcacccaatatatattgctctctgaaaatggtggttaatgaaaattaacttactattttctgacagcaaagaaattgtgctatcagatcgttttagagctagaaatagcaagttaaaataaggctagtccgttatcaacttgaaaaagtggcaccgagtcggtgctttt

SEQ ID NO: 13 - DNA, 40 bp, synthetic construct
  source 1..40: mol_type = other DNA
aatttctactaagtgtagatgcacaaactcggacccactt

SEQ ID NO: 14 - DNA, 42 bp, synthetic construct
  source 1..42: mol_type = other DNA
aatttctactaagtgtagatactttttcacattgataacgga

SEQ ID NO: 15 - DNA, 40 bp, synthetic construct
  source 1..40: mol_type = other DNA
aatttctactaagtgtagatttgataacggactagcctta

SEQ ID NO: 16 - DNA, 25 bp, synthetic construct
  source 1..25: mol_type = other DNA
tctgtttatatattgctgtcaagcg

SEQ ID NO: 17 - DNA, 25 bp, synthetic construct
  source 1..25: mol_type = other DNA
cgcttgacagcaatatataaacaga

SEQ ID NO: 18 - DNA, 25 bp, synthetic construct
  source 1..25: mol_type = other DNA
tagctcttaaagtctgtttatcgcg

SEQ ID NO: 19 - DNA, 25 bp, synthetic construct
  source 1..25: mol_type = other DNA
cgcgataaacagactttaagagcta

SEQ ID NO: 20 - DNA, 24 bp, synthetic construct
  source 1..24: mol_type = other DNA
agaaattgtgctatcagatcagcg

SEQ ID NO: 21 - DNA, 24 bp, synthetic construct
  source 1..24: mol_type = other DNA
cgctgatctgatagcacaatttct

SEQ ID NO: 22 - DNA, 24 bp, synthetic construct
  source 1..24: mol_type = other DNA
tttagcacaaactcggacccactt

SEQ ID NO: 23 - DNA, 24 bp, synthetic construct
  source 1..24: mol_type = other DNA
aagtgggtccgagtttgtgctaaa

SEQ ID NO: 24 - DNA, 26 bp, synthetic construct
  source 1..26: mol_type = other DNA
tttgactttttcacattgataacgga

SEQ ID NO: 25 - DNA, 26 bp, synthetic construct
  source 1..26: mol_type = other DNA
tccgttatcaatgtgaaaaagtcaaa

SEQ ID NO: 26 - DNA, 24 bp, synthetic construct
  source 1..24: mol_type = other DNA
tttgttgataacggactagcctta

SEQ ID NO: 27 - DNA, 24 bp, synthetic construct
  source 1..24: mol_type = other DNA
taaggctagtccgttatcaacaaa

SEQ ID NO: 28 - DNA, 179 bp, synthetic construct
  source 1..179: mol_type = other DNA
ctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaa

SEQ ID NO: 29 - DNA, 3456 bp, synthetic construct
  source 1..3456: mol_type = other DNA
atgggtgttaagccagttactttgtatgacgttgctgaatacgctggagtttcctaccaaactgtctctagagttgttaatcaagcttctcatgtctccgctaagactagagagaaggttgaggctgctatggctgaattgaactatattccaaatagagttgctcagcagttggctggaaagcaatctttgttgattggagtcgctacttcttctttggctttgcatgctccatctcagattgttgctgctattaagtccagagctgaccagttgggagcttctgttgttgtttctatggttgagagatctggagttgaggcttgcaaggctgctgttcataacttgttggctcagagagtttctggattgattattaattacccattggacgatcaagacgctattgccgttgaggccgcttgtaccaacgtcccagctttgttcttggacgtttccgatcaaactccaattaattctattattttttctcacgaggatggaactagattgggagttgaacacttggttgctttgggacatcaacagattgctttgttggctggaccattgtcttccgtttctgctagattgagattggccggatggcacaagtacttgaccagaaaccagattcaaccaattgctgagagagagggagattggtctgctatgtctggattccagcagactatgcagatgttgaacgaaggaattgtcccaaccgctatgttggtcgctaatgaccaaatggctttgggagctatgagagctattactgaatctggattgagagtcggagctgacatttctgttgttggatatgatgacactgaggattcttcttgctacattccaccattgactactattaagcaagacttcagattgttgggacagacttctgttgatagattgttgcagttgtcccaaggacaagctgttaaaggaaaccaattgttgccagtttctttggttaagagaaagactactttggctccaaacactcagactgcttccccaagagctttggctgactctttgatgcaattggctagacaagtctctagattggagtctggacaaggtggcggcggctctgttaacaactccatgaaggatttcttaggcaagaaaacggtggatggagctgatagtctcaatttggccgtgaatctgcaacaacagcagagttcaaacacaattgccaatcaatcgctttcctcaattggattggaaagttttggttacggctctggtatcaaaaacgagtttaacttccaagacttgataggttcaaactctggcagttcagatccgacattttcagtagacgctgacgaggcccaaaaactcgacatttccaacaagaacagtcgtaagagacagaaactaggtttgctgccggtcagcaatgcaacttcccatttgaacggtttcaatggaatgtccaatggaaagtcacactctttctcttcaccgtctgggactaatgacgatgaactaagtggcttgatgttcaactcaccaagcttcaaccccctcacagttaacgattctaccaacaacagcaaccacaatataggtttgtctccgatgtcatgcttattttctacagttcaagaagcatctcaaaaaaagcatggaaattccagtagacacttttcatacccatctgggccggaggacctttggttcaatgagttccaaaaacaggccctcacagccaatggagaaaatgctgtccaacagggagatgatgcttctaagaacaacacagccattcctaaggaccagtcttcgaactcatcgattttcagttcacgttctagtgcagcttctagcaactcaggagacgatattggaaggatgggcccattctccaaaggaccagagattgagttcaactacgattcttttttggaatcgttgaaggcagagtcaccctcttcttcaaagtacaatctgccggaaactttgaaagagtacatgacccttagttcgtctcatctgaatagtcaacactccgacactttggcaaatggcactaacggtaactattctagcaccgtttccaacaacttgagcttaagtttgaactccttctctttctctgacaagttctcattgagtccaccaacaatcactgacgccgaaaagttttcattgatgagaaacttcattgacaacatctcgccatggtttgacacttttgacaataccaaacagtttggaacaaaaattccagttctggccaaaaaatgttcttcattgtactatgccattctggctatatcttctcgtcaaagagaaaggataaagaaagagcacaatgaaaaaacattgcaatgctaccaatactcactacaacagctcatccctactgttcaaagctcaaataatattgagtacattatcacatgtattctcctgagtgtgttccacatcatgtctagtgaaccttcaacccagagggacatcattgtgtcattggcaaaatacattcaagcatgcaacataaacggatttacatctaatgacaaactggaaaagagtattttctggaactatgtcaatttggatttggctacttgtgcaatcggtgaagagtcaatggtcattccttttagctactgggttaaagagacaactgactacaagaccattcaagatgtgaagccatttttcaccaagaagactagcacgacaactgacgatgacttggacgatatgtatgccatctacatgctgtacattagtggtagaatcattaacctgttgaactgcagagatgcgaagctcaattttgagcccaagtgggagtttttgtggaatgaactcaatgaatgggaattgaacaaacccttgacctttcaaagtattgttcagttcaaggccaatgacgaatcgcagggcggatcaacttttccaactgttctattctccaactctcgaagctgttacagtaaccagctgtatcatatgagctacatcatcttagtgcagaataaaccacgattatacaaaatcccctttactacagtttctgcttcaatgtcatctccatcggacaacaaagctgggatgtctgcttccagcacacctgcttcagaccaccacgcttctggtgatcatttgtctccaagaagtgtagagccctctctttcgacaacgttgagccctccgcctaatgcaaacggtgcaggtaacaagttccgctctacgctctggcatgccaagcagatctgtgggatttctatcaacaacaaccacaacagcaatctagcagccaaagtgaactcattgcaaccattgtggcacgctggaaagctaattagttccaagtctgaacatacacagttgctgaaactgttgaacaaccttgagtgtgcaacaggctggcctatgaactggaagggcaaggagttaattgactactggaatgttgaagaataa

SEQ ID NO: 30 - DNA, 468 bp, synthetic construct
  source 1..468: mol_type = other DNA
tctcatgtttgacagcttatcatcgataagctgactcatgttggtattgtgaaatagacgcagatcgggaacgagctcctcgagtgtgtggaattgtgagcggataacaatttcacacagtcgagtgtgtggaattgtgagcggataacaatttcacacagtcgagtgtgtggaattgtgagcggataacaatttcacacagtcgagtgtgtggaattgtgagcggataacaatttcacacagtcgagtgtgtggaattgtgagcggataacaatttcacacagggcccctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaa

SEQ ID NO: 31 - DNA, 100 bp, synthetic construct
  source 1..100: mol_type = other DNA
cttactttcataattgcgacgttttagagctagaaatagcaagttaaaataaggctagtccgttatcaacttgaaaaagtggcaccgagtcggtgctttt

SEQ ID NO: 32 - DNA, 100 bp, synthetic construct
  source 1..100: mol_type = other DNA
aaaaacaactaattattcgagttttagagctagaaatagcaagttaaaataaggctagtccgttatcaacttgaaaaagtggcaccgagtcggtgctttt

SEQ ID NO: 33 - DNA, 100 bp, synthetic construct
  source 1..100: mol_type = other DNA
aaaatcaaaagcttgtcaatgttttagagctagaaatagcaagttaaaataaggctagtccgttatcaacttgaaaaagtggcaccgagtcggtgctttt

SEQ ID NO: 34 - DNA, 42 bp, synthetic construct
  source 1..42: mol_type = other DNA
gagacagcagggctgactaagtcgaccatcatcatcatcatc

SEQ ID NO: 35 - DNA, 75 bp, synthetic construct
  source 1..75: mol_type = other DNA
gacctttctcttcttttttggaggagtgcaacccatactagtcgaaatagttgttcaattgattgaaatagggac

SEQ ID NO: 36 - DNA, 65 bp, synthetic construct
  source 1..65: mol_type = other DNA
caaaaaagaagagaaaggtcatggacaagaagtactccattgggctcgctatcggcacaaacagc

SEQ ID NO: 37 - DNA, 24 bp, synthetic construct
  source 1..24: mol_type = other DNA
cacgatggcatccacgtcgtagtc

SEQ ID NO: 38 - DNA, 22 bp, synthetic construct
  source 1..22: mol_type = other DNA
acgacgtggatgccatcgtgcc

SEQ ID NO: 39 - DNA, 20 bp, synthetic construct
  source 1..20: mol_type = other DNA
ttagtcagccctgctgtctc

SEQ ID NO: 40 - DNA, 44 bp, synthetic construct
  source 1..44: mol_type = other DNA
tccagtgtcgaaaacgagctagatctaacatccaaagacgaaag

SEQ ID NO: 41 - DNA, 54 bp, synthetic construct
  source 1..54: mol_type = other DNA
gcggccgcataggccactagataattagttgttttttgatcttctcaagttgtc

SEQ ID NO: 42 - DNA, 59 bp, synthetic construct
  source 1..59: mol_type = other DNA
cgcgccttaattaacccggggatccctcgagagatcttttttgtagaaatgtcttggtg

SEQ ID NO: 43 - DNA, 104 bp, synthetic construct
  source 1..104: mol_type = other DNA
ctgtttatatattgctgtcagacgagcttactcgtttcgtcctcacggactcatcagtgacagtctagaggtaccatagttgttcaattgattgaaatagggac

SEQ ID NO: 44 - DNA, 109 bp, synthetic construct
  source 1..109: mol_type = other DNA
ggcaccgagtcggtgcttttggccggcatggtcccagcctcctcgctggcgccggctgggcaacatgcttcggcatggcgaatgggacactagtggatgtcagaatgcc

SEQ ID NO: 45 - DNA, 71 bp, synthetic construct
  source 1..71: mol_type = other DNA
gaagcttcgtacgctgcaggtcgacaagcttgcacaaacgaacgtctcacttaatcttctgtactctgaag

SEQ ID NO: 46 - DNA, 40 bp, synthetic construct
  source 1..40: mol_type = other DNA
acttgacagcaatatataaagacgagcttactcgtttcgt

SEQ ID NO: 47 - DNA, 79 bp, synthetic construct
  source 1..79: mol_type = other DNA
ggcaccgagtcggtgcttttggccggcatggtcccagcctcctcgctggcgccggctgggcaacatgcttcggcatggc

SEQ ID NO: 48 - DNA, 40 bp, synthetic construct
  source 1..40: mol_type = other DNA
ttttttttatcatcattattgacgagcttactcgtttcgt

SEQ ID NO: 49 - DNA, 40 bp, synthetic construct
  source 1..40: mol_type = other DNA
gtcgcaattatgaaagtaaggacgagcttactcgtttcgt

SEQ ID NO: 50 - DNA, 40 bp, synthetic construct
  source 1..40: mol_type = other DNA
tcgaataattagttgtttttgacgagcttactcgtttcgt

SEQ ID NO: 51 - DNA, 40 bp, synthetic construct
  source 1..40: mol_type = other DNA
attgacaagcttttgattttgacgagcttactcgtttcgt

SEQ ID NO: 52 - DNA, 46 bp, synthetic construct
  source 1..46: mol_type = other DNA
ttgacgagtacggtggttaacatcatcatcatcatcattgagtttg

SEQ ID NO: 53 - DNA, 27 bp, synthetic construct
  source 1..27: mol_type = other DNA
gtaggagaaacgaatccgccgtatttc

SEQ ID NO: 54 - DNA, 27 bp, synthetic construct
  source 1..27: mol_type = other DNA
ggcggattcgtttctcctacagtcgct

SEQ ID NO: 55 - DNA, 26 bp, synthetic construct
  source 1..26: mol_type = other DNA
tctgcagctcgcgcgcactagcgagc

SEQ ID NO: 56 - DNA, 26 bp, synthetic construct
  source 1..26: mol_type = other DNA
tagtgcgcgcgagctgcagaaaggta

SEQ ID NO: 57 - DNA, 26 bp, synthetic construct
  source 1..26: mol_type = other DNA
gtagacctgtactcctttctgtctat

SEQ ID NO: 58 - DNA, 24 bp, synthetic construct
  source 1..24: mol_type = other DNA
agaaaggagtacaggtctacaaag

SEQ ID NO: 59 - DNA, 53 bp, synthetic construct
  source 1..53: mol_type = other DNA
gaaacgtcggttggtggagcagagccgccgccaccgtcagccctgctgtctcc

SEQ ID NO: 60 - DNA, 50 bp, synthetic construct
  source 1..50: mol_type = other DNA
ctcagaccagcgtgaaacacggtggcggcggctctgctccaccaaccgac

SEQ ID NO: 61 - DNA, 44 bp, synthetic construct
  source 1..44: mol_type = other DNA
aacttctccagcttgctcatgacctttctcttcttttttggagg

SEQ ID NO: 62 - DNA, 21 bp, synthetic construct
  source 1..21: mol_type = other DNA
atgagcaagctggagaagttc

SEQ ID NO: 63 - DNA, 20 bp, synthetic construct
  source 1..20: mol_type = other DNA
tcgcctctggcaatgccgat

SEQ ID NO: 64 - DNA, 21 bp, synthetic construct
  source 1..21: mol_type = other DNA
atcggcattgccagaggcgag

SEQ ID NO: 65 - DNA, 18 bp, synthetic construct
  source 1..18: mol_type = other DNA
gtgtttcacgctggtctg

SEQ ID NO: 66 - DNA, 41 bp, synthetic construct
  source 1..41: mol_type = other DNA
tgacagcaatatataaacagagacgagcttactcgtttcgt

SEQ ID NO: 67 - DNA, 41 bp, synthetic construct
  source 1..41: mol_type = other DNA
ataaacagactttaagagctagacgagcttactcgtttcgt

SEQ ID NO: 68 - DNA, 40 bp, synthetic construct
  source 1..40: mol_type = other DNA
gcaatatatattgggtgacggacgagcttactcgtttcgt

SEQ ID NO: 69 - DNA, 40 bp, synthetic construct
  source 1..40: mol_type = other DNA
atctacacttagtagaaattgacgagcttactcgtttcgt

SEQ ID NO: 70 - DNA, 79 bp, synthetic construct
  source 1..79: mol_type = other DNA
gcacaaactcggacccacttggccggcatggtcccagcctcctcgctggcgccggctgggcaacatgcttcggcatggc

SEQ ID NO: 71 - DNA, 81 bp, synthetic construct
  source 1..81: mol_type = other DNA
actttttcacattgataacggaggccggcatggtcccagcctcctcgctggcgccggctgggcaacatgcttcggcatggc

SEQ ID NO: 72 - DNA, 79 bp, synthetic construct
  source 1..79: mol_type = other DNA
ttgataacggactagccttaggccggcatggtcccagcctcctcgctggcgccggctgggcaacatgcttcggcatggc

SEQ ID NO: 73 - DNA, 40 bp, synthetic construct
  source 1..40: mol_type = other DNA
ttatatattgctgtcaagcgctaacccctacttgacagca

SEQ ID NO: 74 - DNA, 30 bp, synthetic construct
  source 1..30: mol_type = other DNA
ctgatgttactgaaggatcagatcacgcat

SEQ ID NO: 75 - DNA, 30 bp, synthetic construct
  source 1..30: mol_type = other DNA
tgatccttcagtaacatcagagattttgag

SEQ ID NO: 76 - DNA, 59 bp, synthetic construct
  source 1..59: mol_type = other DNA
cgcttgacagcaatatataaacagactcgaggagctcgttcccgatctgcgtctatttc

SEQ ID NO: 77 - DNA, 45 bp, synthetic construct
  source 1..45: mol_type = other DNA
cgcttgacagcaatatataaacagactaacccctacttgacagca

SEQ ID NO: 78 - DNA, 59 bp, synthetic construct
  source 1..59: mol_type = other DNA
tctgtttatatattgctgtcaagcgctcgaggagctcgttcccgatctgcgtctatttc

SEQ ID NO: 79 - DNA, 45 bp, synthetic construct
  source 1..45: mol_type = other DNA
tagctcttaaagtctgtttatcgcgctaacccctacttgacagca

SEQ ID NO: 80 - DNA, 59 bp, synthetic construct
  source 1..59: mol_type = other DNA
cgcgataaacagactttaagagctactcgaggagctcgttcccgatctgcgtctatttc

SEQ ID NO: 81 - DNA, 45 bp, synthetic construct
  source 1..45: mol_type = other DNA
cgcgataaacagactttaagagctactaacccctacttgacagca

SEQ ID NO: 82 - DNA, 59 bp, synthetic construct
  source 1..59: mol_type = other DNA
tagctcttaaagtctgtttatcgcgctcgaggagctcgttcccgatctgcgtctatttc

SEQ ID NO: 83 - DNA, 44 bp, synthetic construct
  source 1..44: mol_type = other DNA
agaaattgtgctatcagatcagcgctaacccctacttgacagca

SEQ ID NO: 84 - DNA, 58 bp, synthetic construct
  source 1..58: mol_type = other DNA
cgctgatctgatagcacaatttctctcgaggagctcgttcccgatctgcgtctatttc

SEQ ID NO: 85 - DNA, 44 bp, synthetic construct
  source 1..44: mol_type = other DNA
cgctgatctgatagcacaatttctctaacccctacttgacagca

SEQ ID NO: 86 - DNA, 58 bp, synthetic construct
  source 1..58: mol_type = other DNA
agaaattgtgctatcagatcagcgctcgaggagctcgttcccgatctgcgtctatttc

SEQ ID NO: 87 - DNA, 44 bp, synthetic construct
  source 1..44: mol_type = other DNA
tttagcacaaactcggacccacttctaacccctacttgacagca

SEQ ID NO: 88 - DNA, 58 bp, synthetic construct
  source 1..58: mol_type = other DNA
aagtgggtccgagtttgtgctaaactcgaggagctcgttcccgatctgcgtctatttc

SEQ ID NO: 89 - DNA, 44 bp, synthetic construct
  source 1..44: mol_type = other DNA
aagtgggtccgagtttgtgctaaactaacccctacttgacagca

SEQ ID NO: 90 - DNA, 58 bp, synthetic construct
  source 1..58: mol_type = other DNA
tttagcacaaactcggacccacttctcgaggagctcgttcccgatctgcgtctatttc

SEQ ID NO: 91 - DNA, 46 bp, synthetic construct
  source 1..46: mol_type = other DNA
tttgactttttcacattgataacggactaacccctacttgacagca

SEQ ID NO: 92 - DNA, 60 bp, synthetic construct
  source 1..60: mol_type = other DNA
tccgttatcaatgtgaaaaagtcaaactcgaggagctcgttcccgatctgcgtctatttc

SEQ ID NO: 93 - DNA, 46 bp, synthetic construct
  source 1..46: mol_type = other DNA
tccgttatcaatgtgaaaaagtcaaactaacccctacttgacagca

SEQ ID NO: 94 - DNA, 60 bp, synthetic construct
  source 1..60: mol_type = other DNA
tttgactttttcacattgataacggactcgaggagctcgttcccgatctgcgtctatttc

SEQ ID NO: 95 - DNA, 44 bp, synthetic construct
  source 1..44: mol_type = other DNA
tttgttgataacggactagccttactaacccctacttgacagca

SEQ ID NO: 96 - DNA, 58 bp, synthetic construct
  source 1..58: mol_type = other DNA
taaggctagtccgttatcaacaaactcgaggagctcgttcccgatctgcgtctatttc

SEQ ID NO: 97 - DNA, 44 bp, synthetic construct
  source 1..44: mol_type = other DNA
taaggctagtccgttatcaacaaactaacccctacttgacagca

SEQ ID NO: 98 - DNA, 58 bp, synthetic construct
  source 1..58: mol_type = other DNA
tttgttgataacggactagccttactcgaggagctcgttcccgatctgcgtctatttc

SEQ ID NO: 99 - DNA, 27 bp, synthetic construct
  source 1..27: mol_type = other DNA
ctatgaccatgattacgaattcgagct

SEQ ID NO: 100 - DNA, 20 bp, synthetic construct
  source 1..20: mol_type = other DNA
tgcctgcaggtcgactctag

SEQ ID NO: 101 - DNA, 40 bp, synthetic construct
  source 1..40: mol_type = other DNA
aaaacaactaattattcgaaggatcctacaccatgggttc

SEQ ID NO: 102 - DNA, 44 bp, synthetic construct
  source 1..44: mol_type = other DNA
ataagctgtcaaacatgagaattaattcttgaagacgaaagggc

SEQ ID NO: 103 - DNA, 35 bp, synthetic construct
  source 1..35: mol_type = other DNA
actggaatgttgaagaataaccgcggcggccgcca

SEQ ID NO: 104 - DNA, 55 bp, synthetic construct
  source 1..55: mol_type = other DNA
gtaactggcttaacacccatggtactagtttcgaataattagttgttttttgatc

SEQ ID NO: 105 - DNA, 30 bp, synthetic construct
  source 1..30: mol_type = other DNA
ttaagtgagatcgagtgtgtggaattgtga

SEQ ID NO: 106 - DNA, 20 bp, synthetic construct
  source 1..20: mol_type = other DNA
gggagaaaggcggacaggta

SEQ ID NO: 107 - DNA, 20 bp, synthetic construct
  source 1..20: mol_type = other DNA
tacctgtccgcctttctccc

SEQ ID NO: 108 - DNA, 37 bp, synthetic construct
  source 1..37: mol_type = other DNA
acacactcgatctcacttaatcttctgtactctgaag

SEQ ID NO: 109 - DNA, 51 bp, synthetic construct
  source 1..51: mol_type = other DNA
ttaattaacccggggatccctcgaggcttaaaggactccatttcctaaaat

SEQ ID NO: 110 - DNA, 46 bp, synthetic construct
  source 1..46: mol_type = other DNA
tcatcagtgacagtctagaggtaccttttctcagttgatttgtttg

SEQ ID NO: 111 - DNA, 51 bp, synthetic construct
  source 1..51: mol_type = other DNA
ttaattaacccggggatccctcgagtcatctaacactttgtatagcacatc

SEQ ID NO: 112 - DNA, 55 bp, synthetic construct
  source 1..55: mol_type = other DNA
tcatcagtgacagtctagaggtacctcttgatatacttgatactgtgttctttga

SEQ ID NO: 113 - DNA, 55 bp, synthetic construct
  source 1..55: mol_type = other DNA
ttaattaacccggggatccctcgagccttgggttattagtagtgtccgttatttt

SEQ ID NO: 114 - DNA, 54 bp, synthetic construct
  source 1..54: mol_type = other DNA
tcatcagtgacagtctagaggtacctgtttgtttgtgtaattgaaagttgttac

SEQ ID NO: 115 - DNA, 50 bp, synthetic construct
  source 1..50: mol_type = other DNA
ttaattaacccggggatccctcgagatgaaagagtgagaggaaagtacct

SEQ ID NO: 116 - DNA, 60 bp, synthetic construct
  source 1..60: mol_type = other DNA
tcatcagtgacagtctagaggtaccttttagatgtagattgttataattgtgtgtttcaa

SEQ ID NO: 117 - DNA, 50 bp, synthetic construct
  source 1..50: mol_type = other DNA
ttaattaacccggggatccctcgagaactgacagaatgactgactcccta

SEQ ID NO: 118 - DNA, 56 bp, synthetic construct
  source 1..56: mol_type = other DNA
tcatcagtgacagtctagaggtaccatttttaggagataaaaattctggggtaaat

SEQ ID NO: 119 - DNA, 59 bp, synthetic construct
  source 1..59: mol_type = other DNA
ttaattaacccggggatccctcgagaataaaaaaacgttatagaaagaaattggactac

SEQ ID NO: 120 - DNA, 56 bp, synthetic construct
  source 1..56: mol_type = other DNA
tcatcagtgacagtctagaggtacctttgttcgattattctccagataaaatcaac

SEQ ID NO: 121 - DNA, 47 bp, synthetic construct
  source 1..47: mol_type = other DNA
ttaattaacccggggatccctcgagatcttttcagcttcatcgtcag

SEQ ID NO: 122 - DNA, 54 bp, synthetic construct
  source 1..54: mol_type = other DNA
tcatcagtgacagtctagaggtaccgatgatttattgaagtttccaaagttgag